(19) United States
(12) Patent Application Publication
(10) Pub. No.: US 2011/0071155 A1
(43) Pub. Date: Mar. 24, 2011

(54) Tricyclic Nitrogen Containing Compounds And Their Use As Antibacterials

(76) Inventors: Carlos Alemparte-Gallardo, Madrid (ES); David Barros-Aguirre, Madrid (ES); Monica Cacho-Izquierdo, Madrid (ES); Jose Maria Fiandor Roman, Madrid (ES); Modesto Jesus Remuinan-Blanco, Madrid (ES)

(21) Appl. No.: 12/993,186

(22) PCT Filed: May 20, 2009
(86) PCT No.: PCT/EP2009/056178

§ 371 Date: Nov. 17, 2010

(30) Foreign Application Priority Data
May 23, 2008 (EP) 08382019.1

(51) Int. Cl. A61K 31/4375; C07D 471/16; A61K 31/4985; A61P 31/06

(52) U.S. Cl. 514/250; 546/84; 544/346; 514/292

(57) ABSTRACT

Compounds of Formula (I) or pharmaceutically acceptable salts or N-oxides thereof:

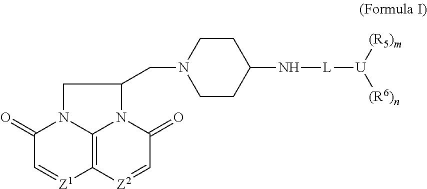

wherein:
one of Z¹ and Z² is CH or N and the other is CH;
L is selected from: —CH₂—CH═CH—, —CH═CH—CH₂—, —(CH₂)ₚ— where p is 2, 3 or 4, —CH₂—CH₂—O—, —O—CH₂—CH₂—, —CH₂—CH₂—NH—, —HN—CH₂—CH₂—, —C(O)—CH═CH—, —CH═CH—C(O)—, —CH₂—C≡C— or —C≡C—CH₂—;
U represents a cyclic group;
m is 0 or 1,
n is independently 0 or 1; and
substituent(s) R⁵ and R⁶ are independently selected from: halo, CF₃, OCF₃, C₁₋₃ alkyl, C₁₋₃ alkoxy, nitro and cyano, pharmaceurtical compositions comprising them, their use in therapy especially against tuberculosis, and methods of preparing them.

### FIELD OF THE INVENTION

This invention relates to compounds, compositions containing them, their use in therapy, including their use as antibacterials, for example in the treatment of tuberculosis, and methods for the preparation of such compounds.

### BACKGROUND OF THE INVENTION

PCT patent publications WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2006002047, WO2006014580, WO2006010040, WO2006017326, WO2006012396, WO2006017468, WO2006020561, WO2006081179, WO2006081264, WO2006081289, WO2006081178, WO2006081182, WO01/25227, WO02/40474, WO02/07572, WO2004024712, WO2004024713, WO2004035569, WO2004087647, WO2004089947, WO2005016916, WO2005097781, WO2006010831, WO2006021448, WO2006032466, WO2006038172, WO2006046552, WO2006099884, WO2006105289, WO2006081178, WO2006081182, WO2007016610, WO2007081597, WO2007071936, WO2007115947, WO2007118130, WO2007122258 and WO2007138974 disclose quinoline, naphthyridine, morpholine, cyclohexane, piperidine and piperazine derivatives and also tricyclic condensed ring compounds, having antibacterial activity. WO2004104000 discloses tricyclic condensed ring compounds capable of selectively acting on cannabinoid receptors.

Synthetic drugs for treating tuberculosis (TB) have been available for over half a century, but incidences of the disease continue to rise world-wide. In 2004, it is estimated that 24,500 people developed active disease and close to 5,500 died each day from TB (World Health Organization, Global Tuberculosis Control: Surveillance, Planning, Financing. WHO Report 2006, Geneva, Switzerland, ISBN 92-4 156314-1). Co-infection with HIV is driving the increase in incidence (Williams, B. G.; Dye, C. Science, 2003, 301, 1535) and the cause of death in 31% of AIDS patients in Africa can be attributed to TB (Corbett, E. L.; Watt, C. J.; Catherine, J.; Walker, N.; Maher D.; Williams, B. G.; Raviglione, M. C.; Dye, C. Arch. Intl. Med., 2003, 163, 1009, Septkowitz, A.; Raffalli, J.; Riley, T.; Kiehn, T. E.; Armstrong, D. Clin. Microbiol. Rev. 1995, 8, 180). When coupled with the emergence of multi-drug resistant strains of Mycobacterium tuberculosis (MDR-TB), the scale of the problem is amplified. It is now more than a decade since the WHO declared TB “a global health emergency” (World Health Organization, Global Tuberculosis Control: Surveillance, Planning, Financing. WHO Report 2006, Geneva, Switzerland, ISBN 92-4 156314-1).

The limitations of tuberculosis therapy and prevention are well known. The current available vaccine, BCG was introduced in 1921 and fails to protect most people past childhood. Patients who do become infected with active disease currently endure combination therapy with isoniazid, rifampin, pyrazinamide and ethambutol for two months and then continue taking isoniazid and rifampin for a further four months. Daily dosing is required and poor compliance drives the emergence and spread of multi-drug-resistant strains, which are challenging to treat. A recently published detailed review discusses many aspects of TB such as pathogenesis, epidemiology, drug discovery and vaccine development to date (Nature Medicine, Vol 13(3), pages 263-312).

Shorter courses of more active agents which can be taken less frequently and which present a high barrier to the emergence of resistance, i.e. agents which are effective against multi-drug resistant strains of TB (MDR-TB), are urgently required. There is therefore a need to discover and develop new chemical entities to treat TB (recent synthetic leads are reviewed in: Ballell, L.; Field, R. A.; Duncan, K.; Young, R. J. Antimicrob. Agents Chemother. 2005, 49, 2153).

### DETAILED DESCRIPTION OF THE INVENTION

In one aspect the present invention provides a compound of Formula (I) or a pharmaceutically acceptable salt or N-oxide thereof:

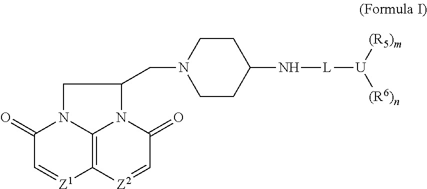

(relative stereochemistry shown)
wherein:

one of Z¹ and Z² is CH or N and the other is CH;

L is selected from: —CH₂—CH═CH—, —CH═CH—CH₂—, —(CH₂)ₚ— where p is 2, 3 or 4, —CH₂—CH₂—O—, —O—CH₂—CH₂—, —CH₂—CH₂—NH—, —HN—CH₂—CH₂—, —C(O)—CH═CH—, —CH═CH—C(O)—, —CH═CH, or —C≡C—;

U represents a cyclic group selected from: phenyl, pyridyl, pyridazinyl, pyrimidinyl, pyrazinyl, thiazolyl, furanyl, imidazolyl and thiophenyl;

m is 0 or 1,

n is independently 0 or 1; and

substituent(s) R⁵ and R⁶ are independently selected from: halo, CF₃, OCF₃, C₁₋₃ alkyl, C₁₋₃ alkoxy, nitro and cyano.

In one embodiment the compound of Formula (I) may be a compound of Formula (IA) or a pharmaceutically acceptable salt or N-oxide thereof:

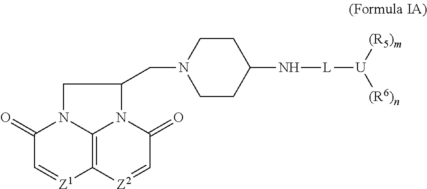

(relative stereochemistry shown)
wherein:

one of Z¹ and Z² is CH or N and the other is CH;

L is selected from: —CH₂—CH═CH—, —CH═CH—CH₂—, —(CH₂)ₚ— where p is 2, 3 or 4, —CH₂—CH₂—O—, —O—CH₂—CH₂—, —CH₂—CH₂—NH—, —HN—CH₂—CH₂—, —C(O)—CH═CH— or —CH═CH—C(O)—;

U represents a cyclic group selected from: phenyl, pyridyl, pyridazinyl, pyrimidinyl, pyrazinyl, thiazolyl, furanyl, imidazolyl and thiophenyl;

m is 0 or 1,

n is independently 0 or 1; and

substituent(s) R⁵ and R⁶ are independently selected from: halo, CF₃, OCF₃, C₁₋₃ alkyl, C₁₋₃ alkoxy, nitro and cyano.

The invention further provides a pharmaceutical composition comprising a compound of Formula (I), or a pharmaceutically acceptable salt or N-oxide thereof, and one or more pharmaceutically acceptable carriers, excipients or diluents.

The invention also provides a method of treatment of tuberculosis in mammals, particularly in man, which method comprises the administration to a mammal in need of such treatment an effective amount of a compound of Formula (I), or a pharmaceutically acceptable salt or N-oxide thereof.

This invention further provides a method of treatment of bacterial infections in mammals, particularly in man, which method comprises the administration to a mammal in need of such treatment an effective amount of a compound of Formula (I), or a pharmaceutically acceptable salt or N-oxide thereof.

The invention further provides a compound of Formula (I), or a pharmaceutically acceptable salt or N-oxide thereof, for use in therapy.

The invention yet further provides a compound of Formula (I), or a pharmaceutically acceptable salt or N-oxide thereof, for use in the treatment of tuberculosis in mammals, particularly in man.

The invention yet further provides a compound of Formula (I), or a pharmaceutically acceptable salt or N-oxide thereof, for use in the treatment of bacterial infections in mammals, particularly in man.

The invention still further provides the use of a compound of Formula (I), or a pharmaceutically acceptable salt or N-oxide thereof, in the manufacture of a medicament for use in the treatment of tuberculosis in mammals, particularly in man.

The invention still further provides the use of a compound of Formula (I), or a pharmaceutically acceptable salt or N-oxide thereof, in the manufacture of a medicament for use in the treatment of bacterial infections in mammals, particularly in man.

The invention also provides a pharmaceutical composition comprising a compound of Formula (I), or a pharmaceutically acceptable salt or N-oxide thereof, and one or more pharmaceutically acceptable carriers, excipients or diluents, for use in the treatment of tuberculosis in mammals, particularly in man.

The invention also provides a pharmaceutical composition comprising a compound of Formula (I), or a pharmaceutically acceptable salt or N-oxide thereof, and one or more pharmaceutically acceptable carriers, excipients or diluents, for use in the treatment of bacterial infections in mammals, particularly in man.

Examples of embodiments of Formula (I) are as follows.

In one embodiment cyclic group U is phenyl, 3-pyridyl or 2-furanyl.

In another embodiment linker L, p may be 3.

In another embodiment C₁₋₃ alkoxy is methoxy.

In another embodiment R⁵ is fluorine, m is 1 and n is 0.

In another embodiment m is 1, n is 1 R⁵ is fluorine and R⁶ is also fluorine.

In another embodiment m is 1, n is 0 and R⁵ is NO₂.

In another embodiment m is 1, n is 1 R⁵ is bromine and R⁶ is methoxy.

One or more of the above structural embodiments may be present in a compound of Formula (I).

In an embodiment the absolute stereochemistry of compounds of Formula (I) or a pharmaceutically acceptable salt or N-oxide thereof is indicated by the Formula (IB):

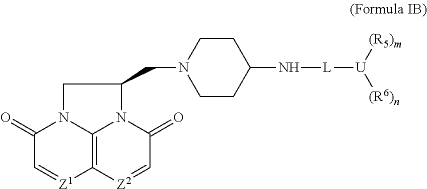

In one aspect, compounds which are useful in the present invention include those mentioned in the examples and their pharmaceutically acceptable salts or N-oxides.

In another aspect, compounds which are useful in the present invention include: (1R)-1-[(4-{[(2E)-3-(2,5-difluorophenyl)-2-propen-1-yl] amino}-1-piperidinyl)methyl]-1,2-dihydro-4H,9H-imidazo[1,2,3-ij]-1,8-naphthyridine-4,9-dione;

2-[(4-{[(2E)-3-(3-fluorophenyl)-2-propen-1-yl]amino}-1-piperidinyl)methyl]-1,2-dihydro-3H,8H-2a,5,8a-triazaacenaphthylene-3,8-dione;

2-[(4-{[3-(3-fluorophenyl)propyl]amino}-1-piperidinyl)methyl]-1,2-dihydro-3H,8H-2a,5,8a-triazaacenaphthylene-3,8-dione;

(1R)-1-[(4-{[(2E)-3-(5-fluoro-3-pyridinyl)-2-propen-1-yl]amino}-1-piperidinyl)methyl]-1,2-dihydro-4H,9H-imidazo[1,2,3-ij]-1,8-naphthyridine-4,9-dione;

2-[(4-{[(2E)-3-(5-fluoro-3-pyridinyl)-2-propen-1-yl]amino}-1-piperidinyl)methyl]-1,2-dihydro-3H,8H-2a,5,8a-triazaacenaphthylene-3,8 -dione;

2-{[4-({2-[(3-fluorophenyl)oxy]ethyl}amino)-1-piperidinyl]methyl}-1,2-dihydro-3H,8H-2a,5,8a-triazaacenaphthylene-3,8-dione;

2-{[4-({2-[(3-fluorophenyl)amino]ethyl}amino)-1-piperidinyl]methyl}-1,2-dihydro-3H,8H-2a,5,8a-triazaacenaphthylene-3,8-dione;

(1R)-1-{[4-({2-[(3-fluorophenyl)amino]ethyl}amino)-1-piperidinyl]methyl}-1,2-dihydro-4H,9H-imidazo[1,2,3-ij]-1,8-naphthyridine-4,9-dione;

2-[(4-{[(2E)-3-(2-furanyl)-2-propen-1-yl]amino}-1-piperidinyl)methyl]-1,2-dihydro-3H,8H-2a,5,8a-triazaacenaphthylene-3,8-dione;

2-{[4-({(2E)-3-[5-bromo-2-(methyloxy)-3-pyridinyl]-2-propen-1-yl}amino)-1-piperidinyl]methyl}-1,2-dihydro-3H,8H-2a,5,8a-triazaacenaphthylene-3,8 -dione;

2-[(4-{[(2E)-3-(2-nitrophenyl)-2-propen-1-yl]amino}-1-piperidinyl)methyl]-1,2-dihydro-3H,8H-2a,5,8a-triazaacenaphthylene-3,8-dione;

2-[(4-{[(2E)-3-phenyl-2-propen-1-yl]amino}-1-piperidinyl)methyl]-1,2-dihydro-3H,8H-2a,5,8a-triazaacenaphthylene-3,8-dione;

(2E)-3-(2,5-difluorophenyl)-N-{1-[(3,8-dioxo-1,2-dihydro-3H,8H-2a,5,8a-triazaacenaphthylen-2-yl)methyl]-4-piperidinyl}-2-propenamide;

2-[(4-{[(3-fluorophenyl)ethynyl]amino}-1-piperidinyl)methyl]-1,2-dihydro-3H,8H-2a,5,8a-triazaacenaphthylene-3,8-dione;

2-[(4-{[(2Z)-3-(3-fluorophenyl)-2-propen-1-yl]amino}-1-piperidinyl)methyl]-1,2-dihydro-3H,8H-2a,5,8a-triazaacenaphthylene-3,8 -dione;

2-[(4-{[2-(3,4-dichlorophenyl)ethyl]amino}-1-piperidinyl)methyl]-1,2-dihydro-3H,8H-2a,5,8a-triazaacenaphthylene-3,8-dione; and;

(2R)-2-[(4-{[(2E)-3-(5-fluoro-3-pyridinyl)-2-propen-l-yl]amino}-1-piperidinyl)methyl]-1,2-dihydro-3H,8H-2a,5,8a-triazaacenaphthylene-3,8-dione.

Certain of the compounds of Formula (I) may exist in the form of optical isomers, e.g. mixtures of isomers in all ratios, e.g. racemic mixtures. The invention includes all such forms, in particular the pure isomeric forms. For example the invention includes enantiomers. The different isomeric forms may be separated or resolved one from the other by conventional methods, or any given isomer may be obtained by conventional synthetic methods or by stereospecific or asymmetric syntheses.

### Terms and Definitions

The term “C₁₋₃ alkyl” as used herein refers to a straight or branched chain alkyl group having 1 to 3 carbon atoms. Examples of C₁₋₃ alkyl groups include methyl, ethyl, n-propyl, iso-propyl.

The term “halo” as used herein refers to fluoro, chloro, bromo and iodo groups. In one aspect, the term “halo” as used herein refers to fluoro, chloro and bromo. An embodiment of halo is fluoro.

The term “C₁₋₃ alkoxy” as used herein refers to a straight or branched chain alkoxy group having 1 to 3 carbon atoms. Examples of C₁₋₃ alkoxy groups include, methoxy, ethoxy, propoxy and isopropoxy.

The term “compounds of the invention” as used herein means a compound of Formula (I) or a pharmaceutically acceptable salt or N-oxide thereof. The term “a compound of the invention” means any one of the compounds of the invention as defined above.

Furthermore, it will be understood that phrases such as “a compound of Formula (I) or a pharmaceutically acceptable salt or N-oxide thereof” or “compounds of the invention” are intended to encompass the compound of Formula (I), a pharmaceutically acceptable salt or N-oxide of the compound of Formula (I), or any pharmaceutically acceptable combination of these. Thus by way of non-limiting example used here for illustrative purpose, “a compound of Formula (I) or a pharmaceutically acceptable salt or N-oxide thereof” encompasses a pharmaceutically acceptable salt of a compound of Formula (I) which is present as a solvate, or this phrase may include a mixture of a compound of Formula (I) and a salt of a compound of Formula (I).

It will be further appreciated that all crystalline forms, polymorphs and enantiomers of the compounds of the invention, or mixtures thereof, are contemplated to be within the scope of the present invention. Unless otherwise specified (for example when the absolute stereochemistry is shown), for compounds of the invention which possesses stereocentres and which can therefore form enantiomers, the compound contains a 1:1 mixture of enantiomers, i.e. a racemic mixture of enantiomers. These may be separated using conventional techniques such as chiral HPLC.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Since the compounds of Formula (I) are intended for use in pharmaceutical compositions it will readily be understood that in particular embodiments they are provided in substantially pure form, for example at least 60% pure, more suitably at least 75% pure and particularly at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and more particularly from 10 to 59% of a compound of Formula (I) or pharmaceutically acceptable salt or N-oxide thereof.

Pharmaceutically acceptable salts of the compounds of Formula (I) include the acid addition or quaternary ammonium salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids. In one aspect of the invention, the salt of a compound of Formula (I) is the hydrochloride salt. In another aspect, the salt of a compound of Formula (I) is the dihydrochloride salt. Compounds of Formula (I) may also be prepared as the N-oxide. The invention extends to all such salts and N-oxides.

Compound Preparation

In further aspects of the invention there are provided processes for preparing compounds of Formula (I) and pharmaceutically acceptable salts or N-oxides thereof.

In one aspect a suitable process comprises the reaction between an amine compound of Formula (IIA) and a compound of Formula (IIB):

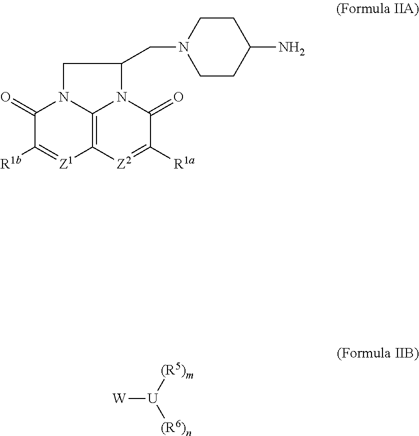

wherein Z¹, Z², L, U, m, n, R⁵ and R⁶ are as defined in Formula (I), and W is a moiety which can react with the —NH₂ group to form the moiety —NH-L-. Such moieties will be apparent to those shilled in the art and include for example i) an aldehyde moiety O═CH—, the reaction being a reductive amination reaction, typically in the presence of a reducing agent such as sodium borohydride, and typically in a dichloromethane/methanol solvent at ambient temperature; or ii) a methane sulphonate moiety, typically initially at ambient temperature then at elevated temperature; or (iii) a carboxylic acid, or an acylating derivative of a carboxylic acid.

See for example Smith, M. B.; March, J. M. Advanced Organic Chemistry, Wiley-Interscience.

Compounds of Formula (IIA) can be made via various preparation schemes.

One such scheme, suitable for compounds in which Z¹ is CH and Z² is N is Scheme (1) below.

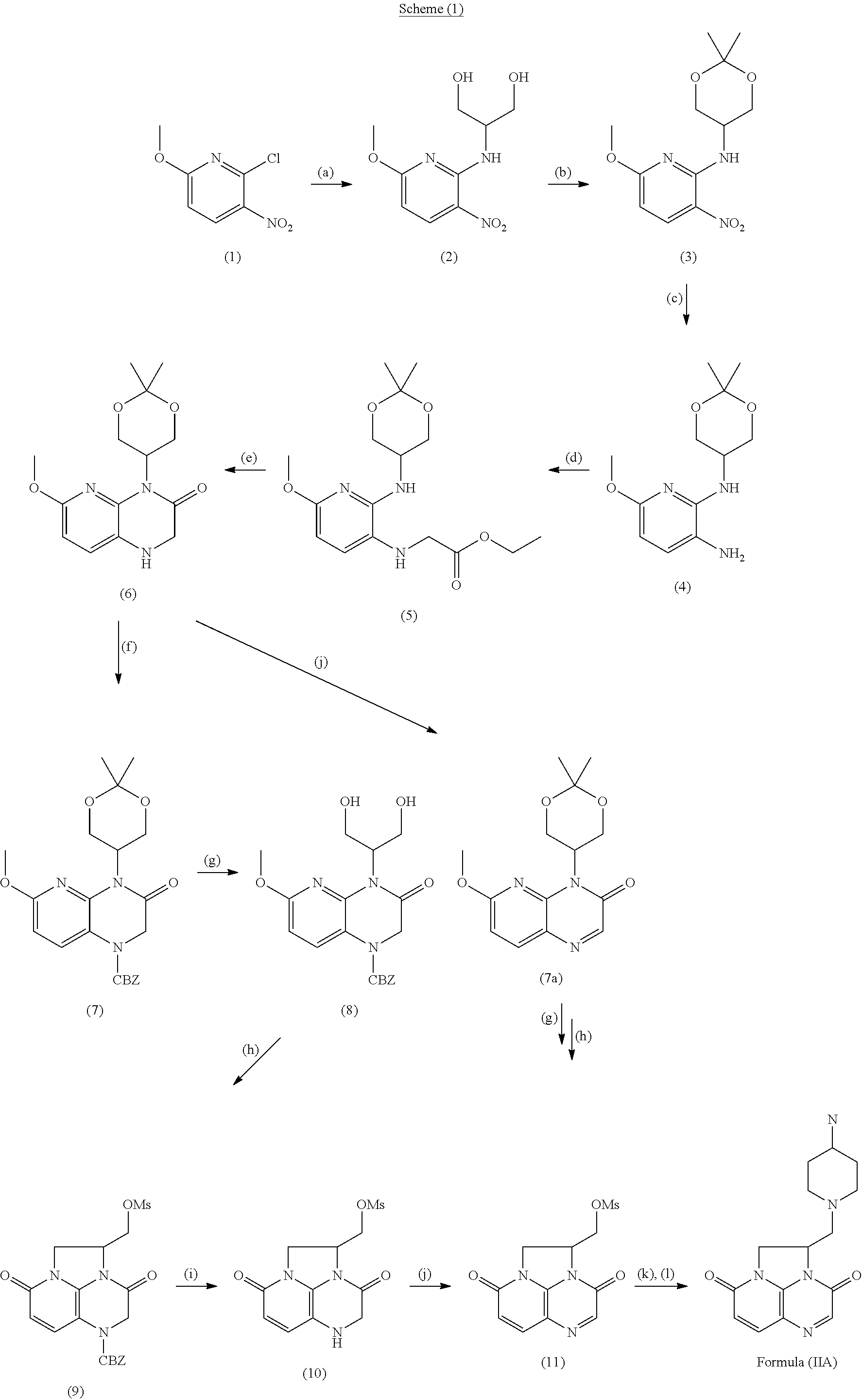

(a) 2-amino-1,3-propanediol (b) 2,2-dimethoxypropane, p-toluenesulfonic acid (c) hydrogen, palladium/charcoal (d) ethyl bromoacetate, K₂CO₃ (e) sodium hydride (f) benzyl chloroformate (g) aqueous acid (h) methane sulphonic anhydride (i) hydrogen, palladium/charcoal (j) MnO₂ (k) 4-(N-tert-butoxycarbonylamino)piperidine, pyridine (l) HCl in 1,4-dioxane.

Reaction of nitropyridine (1) with 2-amino-1,3-propanediol affords diol (2) which is protected as acetal (3). Reduction of the nitro group gives amine (4) which is alkylated to yield ester (5). Cyclisation can be effected with sodium hydride to give (6). This is protected with a carboxybenzyl (CBz) group (7) then cleaved to give the diol (8). Cyclisation with methanesulphonic anhydride affords the mesylate (9), then hydrogenolysis of the CBz group (10) and subsequent oxidation with manganese(II)oxide gives the key dione intermediate mesylate (11). The order of steps may be changed to go via (7a). Reaction of mesylate (11) with 4-(N-tert-butoxycarbonylamino)piperidine and further deprotection by removal of the carbamate moiety affords desired compound of Formula (IIA).

Reaction with an aldehyde of Formula (IIB) may be carried out in the presence of NaBH(OAc)₃ to yield the compound of Formula (I).

Alternatively enantiomerically pure compounds of Formula (IIA) in which Z¹ is CH and Z² is N could be also obtained by preparing the chiral mesylate intermediate in an enantiomerically pure form as depicted in Scheme (1a) below:

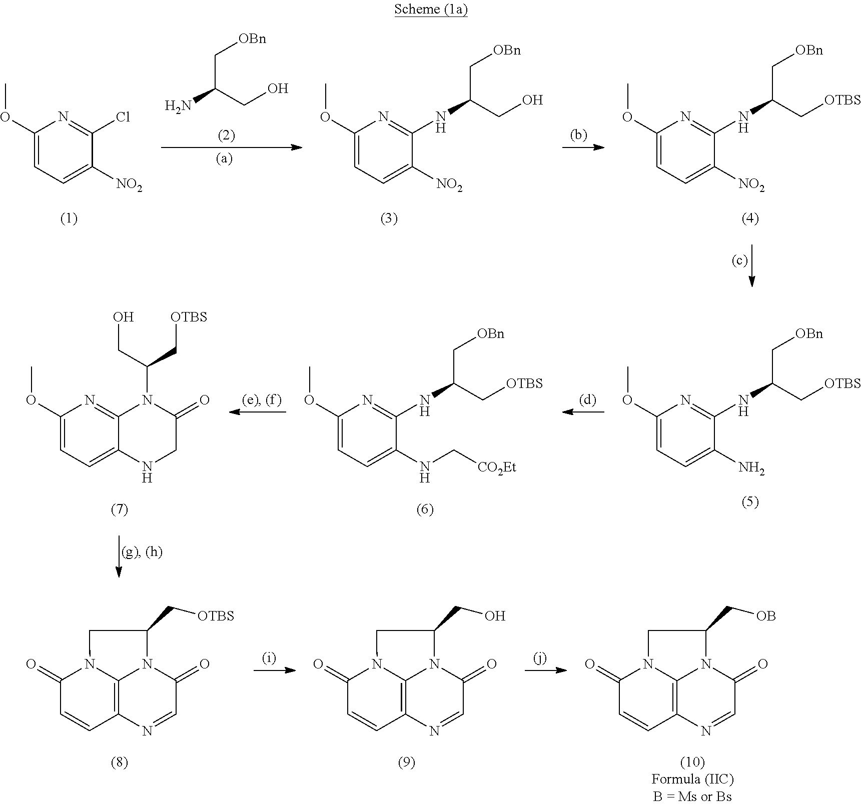

(a) EtOH, reflux, (b) TBS-Cl, (c) Zinc, acetic acid, (d) ethyl bromoacetate, K₂CO₃ (e) NaH, (f) hydrogen, palladium/charcoal,(g) MnO₂, (h) methanesulfonic anhydride (i) TFA, (j) methanesulfonic anhydride or benzenesulfonyl chloride.

Reaction of 2-chloro-6-(methyloxy)-3-nitropyridine with chiral amine (2) gives intermediate (3). Protection of (3) with tert-butyl-dimethylsilylchloride gives (4). Reduction of the nitro group gives amine (5), which is alkylated to yield ester (6).

Cyclisation of (6) can be effected with sodium hydride and then treatment with hydrogen over a palladium/charcoal catalyst gives intermediate (7). Oxidation with manganese (II) oxide and treatment with methanesulfonic anhydride gives (8). This intermediate can be deprotected with TFA to give (9) and reacted with methanesulfonic anhydride or benzenesulfonyl chloride to give (10) as an enantiomercally pure compound. The mesylate or benzenesulfonate (10) formed may then be converted to the amine of Formula (IIa) and finally to the target compound of Formula (I) as generally described herein.

Another scheme, suitable for compounds in which Z¹ is N and Z² is CH, is Scheme (2) below.

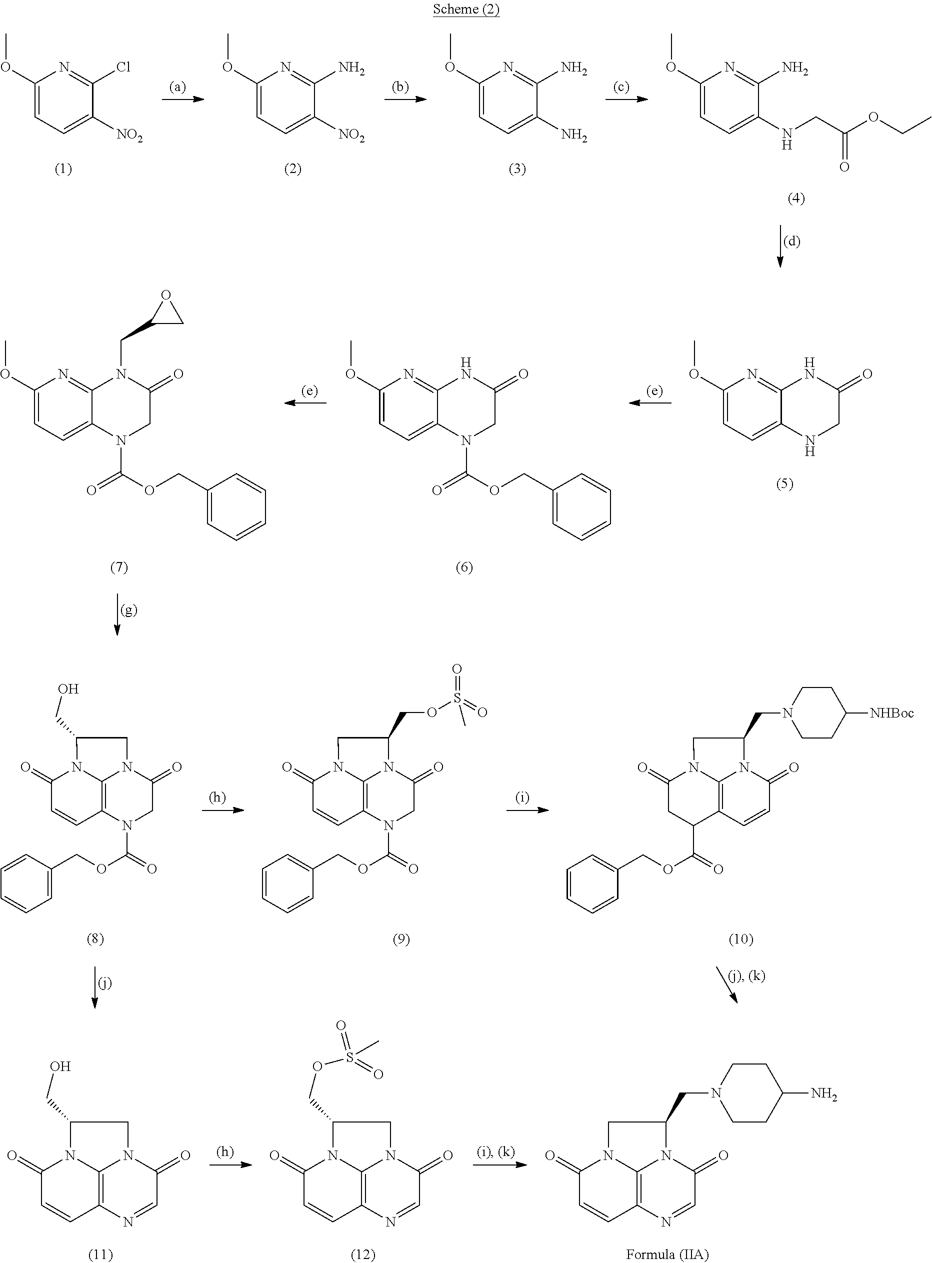

(a) NH₃/MeOH (b) hydrogen, palladium/charcoal (c) ethyl bromoacetate, K₂CO₃ (d) potassium tert-butoxide (e) CBzCl (f) NaH, (S)-glycidyl nosylate (g) DMF, heat (h) methanesulfonyl chloride (i) 4-(N-tert-butoxycarbonylamino)piperidine, pyridine (j) hydrogen, palladium/charcoal then MnO₂ (k) HCl in 1,4-dioxane.

Reaction of nitropyridine (1) with ammonia affords nitro-pyridine (2) which is reduced to bis-aniline (3). Alkylation with ethyl bromoacetate followed by cyclisation with potassium tert-butoxide gives (5). This is protected with a carboxybenzyl group to give (6) which can then be reacted with (commercially available) S-glycidyl nosylate ((2S)-2-oxiranylmethyl 3-nitrobenzenesulfonate) to give (7). Cyclisation under thermal conditions gives (8). Mesylation, displacement with 4-(N-tert-butoxycarbonylamino) piperidine, hydrogenolysis of the CBz group (10) and subsequent oxidation with manganese(II)oxide and deprotection by removal of the carbamate moiety gives the target amine of Formula (IIa). Alternatively hydrogenolysis of the CBz group (11) and subsequent oxidation with manganese(II)oxide, followed by mesylation, displacement with 4-(N-tert-butoxycarbonylamino)piperidine and removed of protective group also gives Formula (IIa). This may be converted to the compound of formula (I) as generally described herein.

Another scheme, suitable for compounds in which Z¹ and Z² are CH, is scheme (3) below.

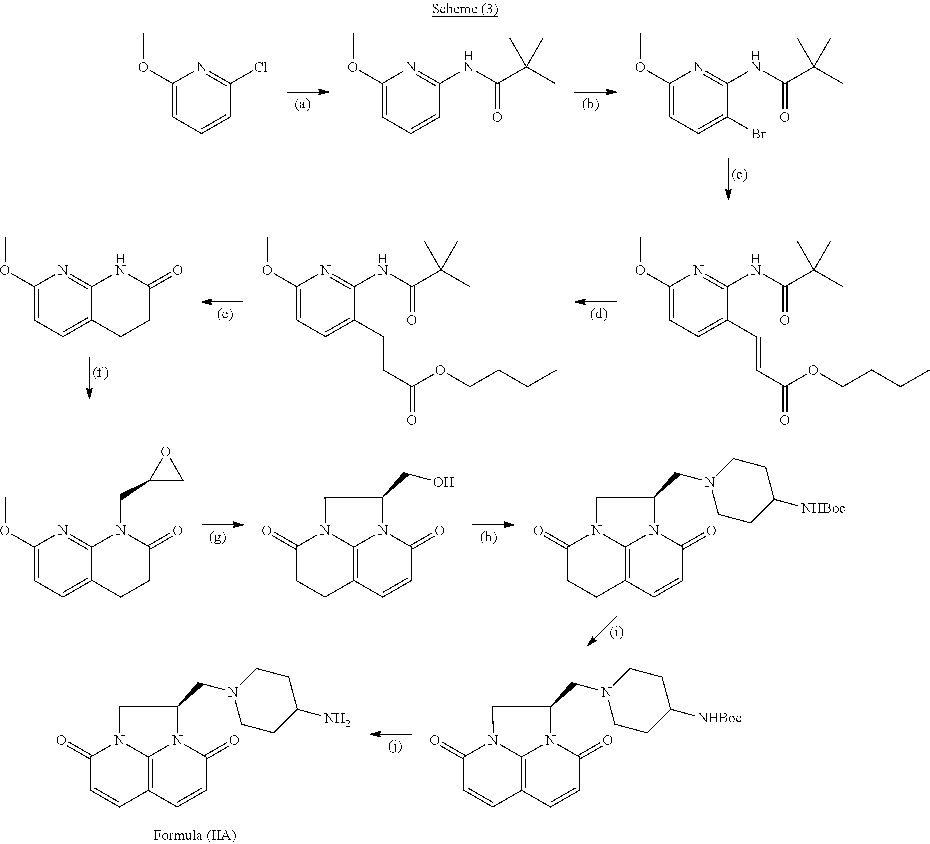

In step (a) trimethylacetamide may be reacted with 2-chloro-6-(methyloxy)pyridine. In step (b) the product of step (a) may be treated with n-butyl lithium and 1,2-dibromoethane. The product from step (b) may be treated in step (c) with n-butyl acrylate. Hydrogenation in the presence of palladium on carbon in step (d) can yield the hydrogenated product. The product of step (d) may be cyclised in step (e) to yield the 3,4-dihydro-1,8-naphthyridin-2(1H)-one by treatment with hydrochloric acid. In step (f) the oxirane may be formed by reaction with sodium hydride then with (2S)-2-oxiranylmethyl 3-nitrobenzenesulfonate. Cyclisation to the imidazonaphthyridine may be done by heating the oxirane, or microwave power. Step (h) to attach the 4-(N-tert-butoxycarbonylamino) piperidine moiety may be performed by formation of the methanesulfonate, then reaction with the corresponding amine. Aromatisation of the ring in step (i) may be done by treatment with DDQ followed by heating. The amine may then be deprotected using acid hydrolysis, step (j) to yield the amine (IIA), which may then be reacted with a compound of Formula IIB e.g. an aldehyde to form the compound of Formula (I).

In another aspect a suitable process for making a compound of Formula (I) comprises the reaction between a compound of Formula (IIC) and a compound of Formula (IID):

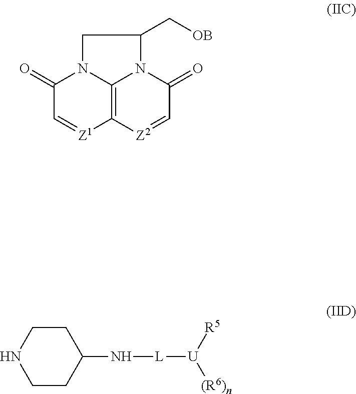

wherein Z¹, Z², U, R⁵ and R⁶ and n are as defined in Formula (I) and B is a benzenesulfonyl moiety. Compounds of formula (IID) may for example be prepared from compounds of formula (IIB) as described above by reaction with a compound of formula (IIE):

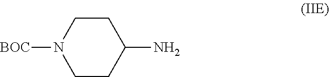

followed by removal of the BOC protecting group from the amino moiety.

Salt forms of compounds of Formula (I) and (IA), e.g. hydrochlorides may be formed by treatment of the corresponding free bases with an acid such as hydrochloric acid, or formation of the compounds in the presence of such an acid.

Many appropriate reagents of Formula (IIB) containing the required R⁵ and optional R⁶ group are known compounds (see for example the commercial sources listed in Table 1) or may be prepared analogously to known compounds. See for example WO02/08224, WO02/50061, WO02/56882, WO02/96907, WO2003087098, WO2003010138, WO2003064421, WO2003064431, WO2004002992, WO2004002490, WO2004014361, WO2004041210,WO2004096982, WO2002050036, WO2004058144, WO2004087145, WO06002047, WO06014580, WO06010040, WO06017326, WO06012396, WO06137485, WO06017468, WO06020561 and EP0559285.

Using these schemes, alternative reagents to provide alternative substituents such as U, R², R⁵, R⁶ etc. will be apparent to those skilled in the art.

Further details for the preparation of compounds of Formula (I) are found in the Examples.

For example some preparative schemes for preparing compounds of Formula (IIB) are as follows.

### Preparative Schemes 4 and 5.

These two schemes are suitable for preparation of compounds of Formula (IIB) for use in preparation of compounds of Formula (I) in which linker L is —CH₂—CH═CH or —(CH₂)₃—.

Preparative Scheme 4 Preparative Scheme 5

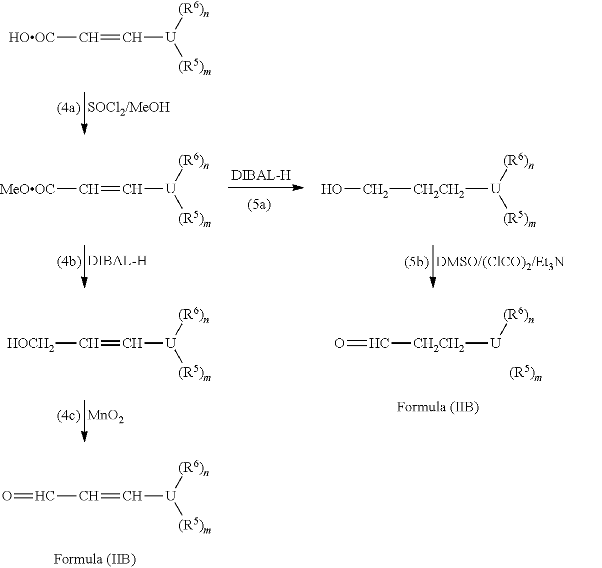

Preparative Scheme 6

This preparative scheme is suitable for preparation of compounds of Formula (IIB) for use in preparation of compounds of Formula (I) in which U is pyridyl and linker L is —CH₂—CH═CH—:

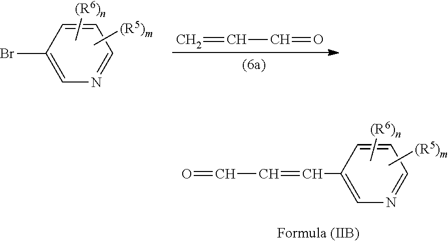

Preparative Scheme 7

This preparative scheme is suitable for preparation of compounds of Formula (IIB) for use in preparation of compounds of Formula (I) in which linker L is —CH₂—CH₂—O—:

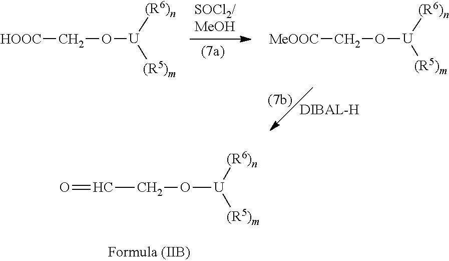

Preparative Scheme 8

This preparative scheme is suitable for preparation of compounds of Formula (IIB) for use in preparation of compounds of Formula (I) in which linker L is —CH₂—CH₂—NH—:

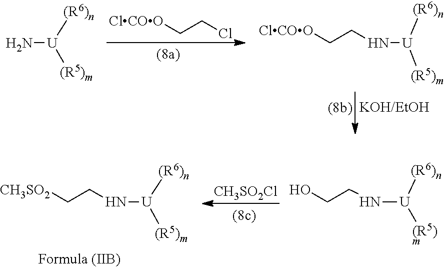

Using these preparative schemes, alternative reagents to provide alternative substituents such as R², R⁵, R⁶ etc. will be apparent to those skilled in the art.

Further details for the preparation of compounds of Formula (I) are found in the examples.

Formulations and Administration

The antibacterial and/or antitubercular compounds according to the invention may be formulated for administration in any convenient way for use in human or veterinary medicine, by analogy with other antibacterials or antitubercular compounds.

The pharmaceutical compositions of the invention include those in a form adapted for oral, topical or parenteral use and may be used for the treatment of bacterial infection or infection with Mycobacterium tuberculosis in mammals including humans.

The composition may be formulated for administration by any route appropriate to antibacterial and/or antitubercular therapy. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Formulations for oral administration may for example comprise tablets or capsules in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Formulations for oral administration may also be in liquid form, for example in the form of aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

Depending on the route of administration the compositions may contain from 0.1% by weight, preferably from 10-60% by weight, of a compound of the invention. Where the compositions comprise dosage units, each unit will preferably contain from 50-1000 mg of the active ingredient. The dosage as employed for adult human treatment will preferably range from 100 to 3000 mg per day, for instance 1500 mg per day depending on the route and frequency of administration. Suitably the dosage is from 5 to 30 mg/kg per day.

The compound of Formula (I), or a pharmaceutically acceptable pharmaceutically acceptable salt or N-oxide thereof, may be the sole therapeutic agent in the compositions of the invention, or it may be present in the formulation in combination with one or more additional therapeutic agents. The invention thus provides, in a further aspect, a combination comprising a compound of Formula (I), or a pharmaceutically acceptable salt or N-oxide thereof together with one or more additional therapeutic agents.

The one or more additional therapeutic agent is, for example, an agent useful for the treatment of tuberculosis in a mammal. Examples of such therapeutic agents include isoniazid, ethambutol, rifampin, pirazinamide, streptomycin, capreomycin, ciprofloxacin and clofazimine.

When a compound of Formula (I), or a pharmaceutically acceptable pharmaceutically acceptable salt or N-oxide thereof is used in combination with one or more additional therapeutic agents, the dose of the compound or agent may differ from that when the compound or agent is used alone. Appropriate doses will be readily appreciated by those skilled in the art. It will be appreciated that the amount of a compound of the invention and the one or more additional therapeutic agents required for use in treatment will vary with the nature of the condition being treated and the age and the condition of the patient and will be ultimately at the discretion of the attendant physician or veterinarian.

The combinations may conveniently be presented for use in the form of a pharmaceutical formulation. In a further aspect of the present invention there is provided a pharmaceutical combination comprising a compound of Formula (I), or a pharmaceutically acceptable salt or N-oxide thereof, together with one or more additional therapeutic agents, and one or more pharmaceutically acceptable carriers, excipients or diluents. The individual components of such combinations may be administered either sequentially or simultaneously in separate or combined pharmaceutical formulations by any convenient route.

When administration is sequential, either the compound of the present invention or one or more additional therapeutic agent may be administered first. When administration is simultaneous, the combination may be administered either in the same or different pharmaceutical composition. When combined in the same formulation it will be appreciated that the compound and agents must be stable and compatible with each other and the other components of the formulation. When formulated separately they may be provided in any convenient formulation, conveniently in such manner as are known for such compounds in the art.

The compound of Formula (I) may be the sole therapeutic agent in the compositions of the invention or a combination with one or more other antibacterial and/or antitubercular compound. If the other antibacterial is a β-lactam then a β-lactamase inhibitor may also be employed.

Compounds of Formula (I) may also be used in the treatment of bacterial infections caused by a wide range of organisms including both Gram-negative and Gram-positive organisms. Some compounds of Formula (I) may be active against more than one organism. This may be determined by the methods described herein.

All publications, including but not limited to patents and patent applications, cited in this specification are herein incorporated by reference as if each individual publication were specifically and individually indicated to be incorporated by reference herein as though fully set forth.

The following Examples illustrate the preparation of certain compounds of Formula (I) and the activity of certain compounds of Formula (I) against various bacterial organisms and organisms which are known to cause tuberculosis.

### Examples and Experimental

### General

### Abbreviations in the Examples:

| | |
|---|---|
| EtOAc | ethyl acetate |
| AcOH | acetic acid |
| Ac₂O | acetic anhydride |
| CAN | acetonitrile |
| BOC | N-tert-butoxycarbonyl |
| BOC anhydride | di-tert-butyl dicarbonate |
| Celite ® | a filter aid composed of acid-washed diatomaceous silica, |
| | (a trademark of Manville Corp., Denver, Colorado) |
| DME | dimethoxyethane |
| DCM | dichloromethane |
| DDQ | 2,3-dichloro-5,6-dicyano-1,4-benzoquinone (an |
| | aromatization reagent) |
| DIBAL-H | diisobutyl aluminium hydride |
| DMF | dimethylformamide |
| DMSO-d6 | deuterated dimethylsulfoxide |
| DMSO | dimethylsulfoxide |
| ES MS | Electrospray mass spectrometry |
| EtOH | ethanol |
| Et₃N | triethylamine |
| h | hours |
| HPLC | high performance liquid chromatography |
| LCMS | Liquid chromatography mass spectroscopy |
| MeOH | methanol |
| MP-carbonate | macroporous triethylammonium methylpolystyrene |
| | carbonate (Argonaut Technologies). |
| NaBH(OAc)₃ | sodium triacetoxyborohydride |
| NMP | N-methyl pyrrolidone (solvent) |
| NMR | Nuclear Magnetic Resonance spectroscopy |
| Pd/C | palladium on carbon catalyst |
| Pd₂(dba)₃ | tris-(dibenxylideneacetone) dipalladium |
| rt | room temperature |
| SCX Cartridge | is an ion exchange column containing strong cation |
| | exchange resin (benzene sulfonic acid) supplied by Varian, |
| | USA. |
| t-BuOMe | methyl t-butyl ether |
| TFA | trifluoroacetic acid |
| THF | tetrahydrofuran |
| uv | ultraviolet |

Proton nuclear magnetic resonance (¹H NMR) spectra were recorded, and chemical shifts are reported in parts per million (δ) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, app=apparent, br=broad. Mass spectra were obtained using electrospray (ES) ionization techniques. All temperatures are reported in degrees centigrade.

As will be understood by the skilled chemist, references to preparations carried out in a similar manner to, or by the general method of, other preparations, may encompass variations in routine parameters such as time, temperature, workup conditions, minor changes in reagent amounts etc.

Reactions involving metal hydrides including lithium hydride, lithium aluminium hydride, di-isobutylaluminium hydride, sodium hydride, sodium borohydride and sodium triacetoxyborohydride are carried out under argon.

### Preparation 1

(1R)-1-[(4-Amino-1-piperidinyl)methyl]-1,2-dihydro-4H,9H-imidazo[1,2,3-ij]-1,8-naphthyridine-4,9- dione dihydrochloride. (Preparitive Scheme 1).

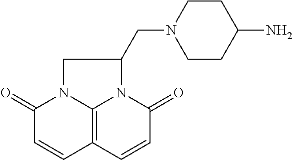

(a) 2,2-Dimethyl-N-[6-(methyloxy)-2-pyridinyl]propanamide

A suspension of trimethylacetamide (18.08 g, 178.744 mmol), Cs₂CO₃ (68.823 g, 211.242 mmol), Pd₂(dba)₃ (1.488 g, 1.625 mmol) and Xantphos (4,5-bis-(diphenylphosphino)-9,9-dimethylxanthene) (1.880 g, 3.249 mmol) in dry, degassed 1,4-dioxane (800 ml) under argon was sonicated for 0.25 h and then treated with 2-chloro-6-(methyloxy)pyridine (19.32 ml, 162.494 mmol). The mixture was then heated at reflux for 24 h. The mixture was evaporated, treated with water (1L) and extracted 3×DCM (1L and then 2×500 ml). The organics were dried (MgSO₄), evaporated and chromatographed (50-100% DCM/40-60 Petroleum ether then 0-5% methanol/DCM) to give title compound as a yellow solid (25.191 g, 121.111 mmol, 75%). Impure fractions were recolumed (eluting as above) to give more product (4.990 g, 23.990 mmol, 15%). Total yield of 90%.

### MS (ES+) m/z 209 (MH⁺, 100%).

(b) N-[3-bromo-6-(methyloxy)-2-pyridinyl]-2,2-dimethylpropanamide

A solution of 2,2-dimethyl-N[6-(methyloxy)-2-pyridinyl]propanamide (55.011 g, 264.467 mmol) in THF (450 ml) in a three necked 1L flask with an internal thermometer under argon was cooled to −78° C. and treated with n-butyl lithium (232 ml, 581.847 mmol) over 15 minutes and then allowed to warm to 0° C. and stirred at 0° C. for 7 h. The mixture was then recooled to −78° C. and treated with 1,2-dibromoethane (27.3 ml, 317 mmol) over 10 minutes and then the solution was allowed warm to room temperature and stirred at room temperature for 30 minutes by which time all the solid which had formed dissolved again. Gas was evolved at this stage so a gas bubbler was placed on one of the flasks necks. Water (100 ml) was then carefully added over 10 minutes. Further water (500 ml) was then added and the mixture was extracted with diethyl ether (3×500 ml). The combined organic solvents were then dried (MgSO₄), filtered, evaporated to give the crude product. This was then dissolved in warm ethyl acetate (100 ml) and allowed to stand in the freezer overnight. The resultant solid which crystallised out was filtered off, washed with ice-cooled diethyl ether (20 ml) and dried in vacuo to give product as a white solid (45.660 g, 159.011 mmol, 60% yield). The filtrate was evaporated and the residue was chromatographed (0-25% ethyl acetate/40-60 petroleum ether) to give recovered starting material (7.264 g, 34.9 mmol), and product as a white solid (8.038 g, 27.992 mmol, 10% yield). The product from recrystallisation and chromatography were identical by NMR and LC-MS and so were combined.

### MS (ES+) m/z 287/289 (MH⁺, 100%).

(c) Butyl (2E)-3-[2-[(2,2-dimethylpropanoyl)amino]-6-(methyloxy)-3-pyridinyl]-2-propenoate

A mixture of N-[3-bromo-6-(methyloxy)-2-pyridinyl]-2,2-dimethylpropanamide (78.738 g, 274 mmol), bis(tri-t-butylphosphine)palladium(O) (1 g, 1.957 mmol) and tris(dibenzylideneacetone)dipalladium(O) (0.892 g, 0.974 mmol) in dry, degassed 1,4-dioxane (600 ml) was treated with n-butyl acrylate (47.1 ml, 329 mmol) and dicyclohexylmethylamine (64.5 ml, 302 mmol). The reaction mixture was then heated at 80° C. for 4 h and then at 120° C. for 3 h. The reaction was then evaporated and water (1000 ml) was added and the mixture was extracted with diethyl ether (3×500 ml). The combined organic solvents were then dried (MgSO₄), filtered, evaporated to give the crude product. This was then dissolved in DCM (300 ml) and chromatographed (10-30% ethyl acetate:40-60 petroleum ether) and then dried in vacuo to give product as a white solid (87.412 g, 95%).

### MS (ES+) m/z 335 (MH⁺, 100%).

(d) Butyl 3-[2-[(2,2-dimethylpropanoyl)amino]-6-(methyloxy)-3-pyridinyl]propanoate

A solution of butyl (2E)-3-[2-[(2,2-dimethylpropanoyl)amino]-6-(methyloxy)-3-pyridinyl]-2-propenoate (43.706 g, 131 mmol) in ethanol (450 ml) under argon at rt was treated with palladium on carbon (10% paste) (5.0 g, 47.0 mmol) and then stirred at rt under 1 atmosphere of hydrogen for 90 h. The reaction mixture was then filtered through a thin pad of silica gel, washing the product through with further ethanol (200 ml). The solvent was then evaporated to give product as a yellow solid (43.549, 99%).

### MS (ES+) m/z 337 (MH⁺, 100%).

(e) 7-(Methyloxy)-3,4-dihydro-1,8-naphthyridin-2(1H)-one

A mixture of butyl 3-[2-[(2,2-dimethylpropanoyl)amino]-6-(methyloxy)-3-pyridinyl]propanoate (86.01 g, 256 mmol) in hydrochloric acid (500 ml, 3000 mmol) (6M aqueous), was heated at 80° C. for 6 h. Reaction was cooled, treated with water (500 ml), transferred to a 5L conical flask and carefully neutralised with solid potassium carbonate (requires around 250 g) (much effervescence was observed). The mixture was then extracted with 20% MeOH/DCM (3×500 ml). The combined organic solvents were then dried (MgSO₄), filtered, evaporated to give the crude product as a yellow solid (35.84 g, 79%).

### MS (ES+) m/z 179 (MH⁺, 100%).

(f) 7-(Methyloxy)-1-[(2R)-2-oxiranylmethyl]-3,4-dihydro-1,8 -naphthyridin-2(1H)-one

A solution of 7-(methyloxy)-3,4-dihydro-1,8-naphthyridin-2(1H)-one (4.974 g, 27.9 mmol) in DMF (100 ml) at 0° C. under argon was treated with sodium hydride (60%, 1.340 g, 33.5 mmol) and allowed to stir at 0° C. for 20 min. The reaction mixture was then treated with (2S)-2-oxiranylmethyl 3-nitrobenzenesulfonate (7.60 g, 29.3 mmol) and then then allowed warm slowly to rt and stirred at rt for 1 h. Water (5 ml) was then added. Reaction was evaporated, saturated aqueous bicarbonate (500 ml) was then added and the mixture was extracted with DCM (3×500 ml). The combined organic solvents were then dried (MgSO₄), filtered, evaporated to give the crude product.

### MS (ES+) m/z 235 (MH⁺, 100%).

(g) (1S)-1-(hydroxymethyl)-1,2,5,6-tetrahydro-4H,9H-imidazo[1,2,3-ij]-1,8-naphthyridine-4,9-dione.

A solution of 7-(methyloxy)-1-[(2R)-2-oxiranylmethyl]-3,4-dihydro-1,8-naphthyridin-2(1H)-one (1.167 g, 4.98 mmol) in DMF (20 ml) under argon was heated to 120° C. for 6 h. Reaction was then evaporated and chromatographed (0-20% methanol/DCM) to give product as an orange solid (339 mg, 31%).

### MS (ES+) m/z 221 (MH⁺, 100%).

Alternatively the reaction can be heated with microwave power at 160° C. for 40 mins.

(h) 1,1-Dimethylethyl (1-{[(2R)-4,9-dioxo-1,2,8,9-tetrahydro-4H,7H-imidazo[1,2,3-ij]-1,8-naphthyridin-2-yl]methyl}-4-piperidinyl)carbamate.

A solution of (15)-1-(hydroxymethyl)-1,2,5,6-tetrahydro-4H,9H-imidazo[1,2,3-ij]-1,8-naphthyridine-4,9-dione (1.909 g, 8.67 mmol) in DCM (100 ml) at 0° C. under argon was treated with triethylamine (1.450 ml, 10.40 mmol) and then methanesulfonyl chloride (0.743 ml, 9.54 mmol) and then allowed to warm to rt and stirred at rt for 1 h. The reaction mixture was then treated with saturated aqueous bicarbonate (100 ml) and the mixture was extracted with DCM (2×100 ml). The combined organic solvents were then dried (MgSO₄), filtered, evaporated to give the crude intermediate [(25)-4,9-dioxo-1,2,8,9-tetrahydro-4H,7H-imidazo[1,2,3-ij]-1,8-naphthyridin-2-yl]methyl methanesulfonate. This was dissolved in dry acetonitrile (100 ml) and then treated with pyridine (1.402 ml, 17.34 mmol) and 1,1-dimethylethyl 4-piperidinylcarbamate (3.47 g, 17.34 mmol) and heated at 70° C. for 20 h. After 20 h more 1,1-dimethylethyl 4-piperidinylcarbamate (3.47 g, 17.34 mmol) and pyridine (1.402 ml, 17.34 mmol) were added and the temperature was increased to reflux (heating block 95° C.) and reaction was stirred at this temperature for a further 4 h. The reaction mixture was then evaporated, treated with saturated aqeous NaHCO₃ (200 ml) was then added and the mixture was extracted with DCM (3×200 ml). The combined organic solvents were then dried (MgSO₄), filtered, evaporated to give the crude product as a brown solid.

### MS (ES+) m/z 403 (MH⁺, 100%).

(i) 1,1-Dimethylethyl (1-{[(1R)-4,9-dioxo-1,2-dihydro-4H,9H-imidazo[1,2,3-ij]-1,8-naphthyridin-1-yl]methyl}-4-piperidinyl)carbamate

A solution of 1,1-dimethylethyl (1-{[(2R)-4,9-dioxo-1,2,8,9-tetrahydro-4H,7H-imidazo[1,2,3-ij]-1,8-naphthyridin-2-yl]methyl}-4-piperidinyl)carbamate (5.710 g, 14.19 mmol) in 1,4-dioxane (50 ml) at rt was treated with DDQ (4.83 g, 21.28 mmol) and then heated at 120° C. for 1 h. The reaction was then cooled to rt. The reaction mixture was treated with saturated aqueous K₂CO₃ (5%, 1000 ml) and extracted with DCM (3×500 ml). The combined organic solvents were then dried (MgSO₄), filtered, evaporated to give the crude product as a brown solid. The reaction was repeated using a further portion of carbamate (2.889 g, 7.18 mmol) in 1,4-dioxane (50 ml) with DDQ (2.444 g, 10.77 mmol). The reaction was performed and worked up as above and the combined residues were chromatographed (0-100% ethyl acetate:40-60 Petroleum ether then 0-20% methanol:ethyl acetate) to give the product as a brown solid (1.532 g, 18%).

### MS (ES+) m/z 401 (MH⁺, 100%).

(j) Title compound: (1R)-1-[(4-Amino-1-piperidinyl)methyl]-1,2-dihydro-4H,9H-imidazo[1,2,3-ij]-1,8-naphthyridine-4,9-dione dihydrochloride

A solution of 1,1-dimethylethyl (1-{[(1R)-4,9-dioxo-1,2-dihydro-4H,9H-imidazo[1,2,3-ij]-1,8-naphthyridin-1-yl]methyl}-4-piperidinyl)carbamate (1.532 g, 3.83 mmol) in chloroform (20 ml) under argon at rt was treated with 4M HCl in 1,4-dioxane (10 ml, 40.0 mmol) and stirred at rt for 0.25 h. Methanol (20 ml) was then added and reaction was stirred for a further 0.25 h. The reaction was then evaporated and triturated with diethyl ether (20 ml). The solid was then dried in vacuo to give the impure product as a brown solid (1.443 g, 101%).

### MS (ES+) m/z 301 (MH⁺, 100%)

1-[(4-Amino-1-piperidinyl)methyl]-1,2-dihydro-4H,9H-imidazo [1,2,3-ij]-1,8-naphthyridine-4,9-dione dihydrochloride made by this general method (Example 5(a)-(j)) was analyzed via chiral HPLC (Chiralpak AS-H (5 microns) and found to be a single enantiomer, presumed to be R.

### Preparation 2

2-[(4-Amino-1-piperidinyl)methyl]-1,2-dihydro-3H,8H-2a,5,8a-triazaacenaphthylene-3,8-dione. (Preparitive Scheme 2)

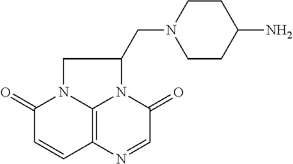

(a) 2-{[6-(Methyloxy)-3-nitro-2-pyridinyl]amino}-1,3-propanediol.

6-Methoxy-2-chloro-3-nitropyridine (36.94 g, 195.9 mmol) and 2-aminopropane-1,3-diol (35.65 g, 391.3 mmol, 2 eq.) were stirrred in ethanol (500 ml) at reflux under argon for 3 hours. The mixture was allowed to cool to room temperature and left overnight. The solvent was partially removed under reduced pressure (to ca. 150 ml) and the resulting bright yellow slurry was poured into ice-water (1.5L) with vigorous stirring. The mixture was stirred for 1 hour then filtered with suction while cold. The solid was washed with ice-cold water (200 ml) and air-dried to give the title compound as a bright yellow solid (45.03 g, 94%). LCMS showed desired product (93%) plus 7% starting material. The product was used without further purification.

### MS (ES+) m/z 244 (MH⁺)

(b) N-(2,2-Dimethyl-1,3-dioxan-5-yl)-6-(methyloxy)-3-nitro-2-pyridinamine

2-{[6-(Methyloxy)-3-nitro-2-pyridinyl]amino}-1,3-propanediol (53.93 g, 228.7 mmol) was stirred in 2,2-dimethoxypropane (900 ml) under argon and p-toluenesulphonic acid monohydrate (1.00 g) was added. The mixture was stirred at room temperature overnight to give a clear yellow solution. This was with diluted with dichloromethane (1L) and the resulting solution was treated with saturated aqueous sodium hydrogen carbonate (20 ml) and solid sodium hydrogen carbonate (20 g) with vigorous stirring (effervescence). The mixture was vigorously stirred for 20 minutes, then the remaining water was absorbed by addition of anhydrous sodium sulphate. The mixture was filtered with suction and the solids were washed with DCM (500 ml). The combined filtrate plus washings were evaporated under reduced pressure to give a yellow solid which was stirred with petroleum ether) (40-60° over the weekend. The solid was isolated by filtration with suction , washed with petroleum ether)(40-60° and air-dried to give the title compound as a bright yellow solid 57.83 g, 92%).

### MS (ES+) m/z 284 (MH⁺).

(c) N²-(2,2-Dimethyl-1,3-dioxan-5-yl)-6-(methyloxy)-2,3-pyridinediamine

N-(2,2-Dimethyl-1,3-dioxan-5-yl)-6-(methyloxy)-3-nitro-2-pyridinamine (35.00 g, 123.6 mmol) was divided into 2 aliquots, each of which was taken up in 1,4-dioxane (500 ml) and hydrogenated over 10% Pd on carbon (paste, 1:1 w:w with water, 4.00 g) under 1 atm. hydrogen pressure, at room temperature for 18 hours. The mixtures were filtered with suction though Celite, using an argon blanket and taking care to minimise contact of the product with air. The solids were washed with 1,4-dioxane and the combined filtrate plus washings were evaporated under reduced pressure to give the title compound as a deep purple oil. This was used immediately in the next step.

### MS (ES+) m/z 254 (MH⁺).

(d) Ethyl N-[2-[(2,2-dimethyl-1,3-dioxan-5-yl)amino]-6-(methyloxy)-3-pyridinyl]glycinate

Crude N²-(2,2-dimethyl-1,3-dioxan-5-yl)-6-(methyloxy)-2,3-pyridinediamine (123.6 mmol) was dissolved in anhydrous DMF (500 ml) under argon and anhydrous potassium carbonate (37.56 g, 2.2 eq.) was added, followed by ethyl bromoacetate (12.31 ml, 0.9 eq.). The mixture was stirred at room temperature overnight. The solvent was removed under reduced pressure and the resulting reddish-brown slurry was partitioned between DCM (1.2L) and water (300 ml). The organic phase was separated and washed with water (300 ml), dried over sodium sulphate, filtered and evaporated under reduced pressure to give a dark red oil, this was taken up in a minimum of DCM and purified by column chromatography on silica (eluted with 5%-60% ethyl acetate in petroleum ether) (40-60°). Appropriate fractions were combined and evaporated under reduced pressure to give the title compound as a dark orange oil (35.42 g, 84%).

### MS (ES+) m/z 340 (MH⁺)

(e) 4-(2,2-Dimethyl-1,3-dioxan-5-yl)-6-(methyloxy)-1,4-dihydropyrido[2,3-b]pyrazin-3(2H)-one

Ethyl N-[2-[(2,2-dimethyl-1,3-dioxan-5-yl)amino]-6-(methyloxy)-3-pyridinyl]glycinate (35.42 g, 104.4 mmol) was dissolved in dry THF (500 ml) and the solution was added dropwise over 2 hours to a cooled (0° C.) suspension of sodium hydride (4.173 g of 60% w:w dispersion in oil, 1.00 eq.) in dry THF (500 ml) under argon. During the addition the colour of the suspension changed from orange to green. The mixture was sirred at 0° C. for a further 15 minutes, then allowed to warm to room temperature and stirred at rt for 1 hour. The mixture was cooled to 0° C. and saturated ammonium chloride (15 ml) was added cautiously with vigorous stirring (effervescence observed). After effervescence had ceased, the mixture was allowed to warm to room temperature and stirred for 4 hours then diluted with ethyl acetate (500 ml) and filtered with suction. The solids were washed with ethyl acetate (300 ml) and the combined filtrate plus washings were evaporated under reduced pressure to give a dark brown solid. This was stirred with petroleum ether (40-60°) (500 ml) plus ethyl acetate (20 ml) and filtered with suction to give a lighter brown solid which was washed with petroleum ether) (40-60°) (100 ml) and air-dried to afford the title compound (25.37 g, 82%).

### MS (ES+) m/z 316 (MNa⁺).

(f) 4-(2,2-Dimethyl-1,3-dioxan-5-yl)-6-(methyloxy)pyrido[2,3-b]pyrazin-3(4H)-one

4-(2,2-Dimethyl-1,3-dioxan-5-yl)-6-(methyloxy)-1,4-dihydropyrido[2,3-b]pyrazin-3(2H)-one (25.37 g) and activated manganese dioxide (120 g, ˜15 eq.) were stirred in DCM (500 ml) at room temperature for 2 hours. The mixture was filtered with suction and the solids were washed with DCM (2×100 ml). The combined filtrate plus washings were evaporated under reduced pressure to give a brown foam; this was purified by column chromatography on silica (eluting with 0%-100% ethyl acetate in petroleum ether) (40-60°). Appropriate fractions were combined and evaporated under reduced pressure to give the title compound as a light tan solid (17.40 g, 69%).

### MS (ES+) m/z 314 (MNa⁺).

(g) 4-[2-Hydroxy-1-(hydroxymethyl)ethyl]-6-(methyloxy)pyrido[2,3-b]pyrazin-3(4H)-one

4-(2,2-Dimethyl-1,3-dioxan-5-yl)-6-(methyloxy)pyrido[2,3-b]pyrazin-3(4H)-one (17.40 g, 59.7 mmol) was dissolved in tetrahydrofuran (THF) (220 ml) to give a dark yellow solution. 1M HCl aq. (200 ml) was added (transient blue and green colours appeared in the solution) and the now light yellow solution was stirred at room temperature for 1 hour. The mixture was concentrated to ca. 300 ml on a rotary evaporator using a cold water bath (some solid was precipitated during this procedure) then was stirred vigorously while solid sodium hydrogen carbonate was added in portions (caution: effervescence) until the mixture was ca. pH 8. The resulting yellow solid was collected by filtration with suction, washed with water (2×20 ml) and air-dried to give the title compound as an amorphous yellow solid (13.805 g, 91%).

### MS (ES+) m/z 252 (MH⁺).

(h) (3,8-Dioxo-1,2,5a,8b-tetrahydro-3H,8H-2a,5,8a-triazaacenaphthylen-2-yl)methyl methanesulfonate

In a 1L round-bottomed flask was placed 4-[2-hydroxy-1-(hydroxymethyl)ethyl]-6-(methyloxy)pyrido[2,3-b]pyrazin-3(4H)-one (11.330 g, 45.1 mmol). Anhydrous chloroform (280 ml) was added, followed by triethylamine (31.4 ml, 225 mmol), and methanesulfonic anhydride (31.4 g, 180 mmol) to give a dark yellow-brown solution. During addition of the methanesulphonic anhydride, an exotherm occurred which was sufficient to cause the solvent to boil. The mixture was stirred vigorously at reflux under argon for 4.5 hours. The mixture was allowed to cool to room temperature, diluted with DCM to ca. 600 ml, and washed with water (200 ml). The organic phase was separated, and the aqueous phase was extracted with DCM (2×200 ml). The combined organic extracts were dried over anhydrous sodium sulphate, filtered, and evaporated under reduced pressure to give crude mesylate as a dark brown oil. This was left overnight under 40-60° petrol (200 ml) plus DCM (50 ml). The resulting solid was isolated by filtration with suction, washed with 4:1 petrol:DCM (2×50 ml) and air-dried to give the title compound as a brown amorphous solid (6.950 g, 52%)

### MS (ES+) m/z 332 (MNa⁺), 298 (MH⁺).

(i) 1,1-Dimethylethyl {1-[(3,8-dioxo-1,2-dihydro-3H,8H-2a,5,8a-triazaacenaphthylen-2-yl)methyl]-4-piperidinyl}carbamate

Crude (3,8-dioxo-1,2,5a,8b-tetrahydro-3H,8H-2a,5,8a-triazaacenaphthylen-2-yl)methyl methanesulfonate (6.950 g, 23.38 mmol) was dissolved in dry acetonitrile (200 ml) and the mixture was treated with pyridine (7.55 ml, 94.0 mmol) followed by 1,1-dimethylethyl 4-piperidinylcarbamate (10.30 g, 51.4 mmol). The mixture was stirred at reflux under argon for 3 h then at 50° C. over the weekend. The mixture was then stirred at 90° C. for 2 hours, then the volatiles were removed under reduced pressure and the residue was partitioned between DCM (600 ml) and water (100 ml). The organic phase was separated and the aqueous phase was extracted with DCM (2×200 ml). The combined organic extracts were washed with water (2×100 ml) dried over anhydrous sodium sulphate, filtered and evaporated under reduced pressure to give a dark tan solid; this was taken up in a minimum of 5% MeOH in DCM and applied chromatographed, eluting with 0-10% MeOH in DCM. Appropriate fractions were combined and evaporated under reduced pressure to give the title compound as an amorphous pale tan solid (5.444 g, 56.8%).

### MS (ES+) m/z 424 (MNa⁺), 402 (MH⁺).

(j) 2-[(4-Amino-1-piperidinyl)methyl]-1,2-dihydro-3H,8H-2a,5,8a-triazaacenaphthylene-3,8-dione (Racemic and Enantiomer 1 and 2 synthesis)
Method A(Racemic synthesis):

1,1-Dimethylethyl {1-[(3,8-dioxo-1,2-dihydro-3H,8H-2a,5,8a-triazaacenaphthylen-2-yl)methyl]-4-piperidinyl}carbamate (1.630 g, 4.06 mmol) was suspended in DCM (30 ml) and 4M HCl in 1,4-dioxane (15 ml) was added to give a bright yellow suspension (and gas evolution). The bright yellow mixture was allowed to stand at room temperature for 1 hour. LCMS showed no starting material remaining The solvents were removed under reduced pressure and the residue was dried under reduced pressure overnight to give the dihydrochloride salt of the title compound as an amorphous tan solid (1.760 g (>theoretical yield for the dihydrochloride owing to the presence of residual solvent).

A portion of the crude dihydrochloride (0.513 g) was dissolved in methanol (4 ml) plus water (1 ml) and applied to an SCX column (10 g) (preconditioned with 2 column volumes of methanol). The column was then eluted, under gravity, using (i) methanol (2×50 ml), (ii) 0.5M ammonia in methanol (3×50 ml fractions). Appropriate fractions were combined and evaporated under reduced pressure to give the crude title compound as a tan amorphous solid (410 mg), which contained methanol-insoluble material not apparent by LCMS (possibly ammonium chloride). The product was shaken with methanol (30 ml) and the suspension was filtered. The solid was washed with methanol (20 ml) and the combined filtrate and washings were evaporated under reduced pressure to give the title compound (360 mg, 87%).

### MS (ES+) m/z 302 (MH⁺).

### Method B

1,1-Dimethylethyl {1-[(3,8-dioxo-1,2-dihydro-3H,8H-2a,5,8a-triazaacenaphthylen-2-yl)methyl]-4-piperidinyl}carbamate (9.735 g, 24.25 mmol) was suspended in DCM (90 ml) and 4M HCl in 1,4-dioxane (45 ml) was added to give a bright yellow suspension (and gas evolution). The bright yellow mixture was stirred at room temperature for 1 hour. The solvents were removed under reduced pressure to give the crude dihydrochloride as a bright yellow amorphous solid (10.420 g) containing residual solvent)

The racemic dihydrochloride (10.42 g) was resolved into its two enantiomers by preparative chiral HPLC using a 4 inch Chiralpak AD (20 microns) preparative column with 50:50:0.1 acetonitrile:methanol:isopropylamine as the mobile phase. The alpha value was 3.1 and baseline resolution was observed for all 3 runs. There was no overlap fraction and both enantiomers (as the free bases) were isolated in >99.8 ee each.

(2R)-2-[(4-Amino-1-piperidinyl)methyl]-1,2-dihydro-3H,8H-2a,5,8a-triazaacenaphthylene-3,8-dione (first component eluted): (3.30 g, light beige solid, chiral HPLC: 100% ee).

### MS (ES+) m/z 302 (MH⁺).

Optical rotation: alpha D=−120° (C=1.00, methanol, 21.8° C.).
(2S)-2-[(4-Amino-1-piperidinyl)methyl]-1,2-dihydro-3H,8H-2a,5,8a-triazaacenaphthylene-3,8-dione (second component eluted): (3.30 g, light beige solid, chiral HPLC: 99.8% ee).

### MS (ES+) m/z 302 (MH⁺).

Optical rotation: alpha D=+122° (C=1.00, methanol, 21.8° C.).

### Preparation 3

1-[(4-Amino-1-piperidinyl)methyl]-1,2,5a,8b-tetrahydro-3H,8H-2a,5,8a-triazaacenaphthylene-3,8-dione (IIIA). (Preparitive Scheme 3).

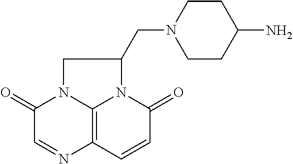

(a) 6-Chloro-3-nitro-N-2-propen-1-yl-2-pyridinamine

This was prepared by a modification of the method of Schmid, S., et al, Synthesis (2005), (18), 3107-3118. A solution of 2,6-dichloro-3-nitropyridine (8.0 g, 41.45 mmol) in anhydrous dichloromethane (180 ml) was cooled to −15° C., under argon. Triethylamine (6.0 ml, 43 mmol) was added and then allylamine (3.23 ml, 43 mmol) was added in small portions over 3 hours, keeping the temperature at −15° C. The reaction mixture was stirred overnight during which time it warmed to room temperature. The reaction mixture was washed with 0.2M aqueous citric acid (100 ml), saturated aqueous NaHCO₃ solution (100 ml), passed through a hydrophobic frit and evaporated to a yellow oil which was purified by chromatography on silica eluting with a 0 to 50% ethyl acetate in hexane giving a yellow solid (7.49 g, 85%).

C₈H₈ClN₃O₂ requires 213, MS (ES+) m/z 214, 216 (MH⁺).
(b) 3-(Bromomethyl)-8-nitro-2,3-dihydroimidazo[1,2-a]pyridin-5(1H)-one

This was prepared by a modification of the method of Schmid, S., et al, Synthesis (2005), (18), 3107-3118. A solution of 6-chloro-3-nitro-N-2-propen-1-yl-2-pyridinamine (20 g, 93.6 mmol) in chlorobenzene (500 ml) was treated with a solution containing bromine (4.75 ml, 92.7 mmol) in chlorobenzene (100 ml), dropwise over 4.5 hours, keeping T<26° C. with cooling when required. The thick suspension was stirred at room temperature for 18 hours and diluted with hexane (200 ml) and then the reaction mixture was then pored into hexane (1000 ml). After 15 minutes the orange precipitate was collected by filtration and washed with hexane (250 ml) to give 26.6 g of an orange solid (3-(bromomethyl)-5-chloro-8-nitro-2,3-dihydroimidazo[1,2-a]pyridin-1-ium bromide). This intermediate was added, over 45 minutes, to a rapidly stirred mixture of saturated aqueous NaHCO₃ solution (1000 ml) and ethyl acetate (500 ml). The bright red mixture was stirred for 1 hour, diluted with ethyl acetate (200 ml) and the layers were separated. The aqueous layer was washed with ethyl acetate (200 ml) and the organic extracts were combined, dried (anhydrous sodium sulphate), filtered and evaporated to give the product as a brown solid (18.3 g, contains 40% 6-bromo-3-(bromomethyl)-8-nitro-2,3-dihydroimidazo[1,2-a]pyridin-5(1H)-one).

C₈H₈BrN₃O₃ requires 273, MS (ES+) m/z 274, 276 (MH⁺).
(c) 1,1-Dimethylethyl {1-[(8-nitro-5-oxo-1,2,3,5-tetrahydroimidazo[1,2-a]pyridin-3-yl)methyl]-4-piperidinyl}carbamate

A suspension of a 3:2 mixture of 3-(bromomethyl)-8-nitro-2,3-dihydroimidazo[1,2-a]pyridin-5(1H)-one and 6-bromo-3-(bromomethyl)-8-nitro-2,3-dihydroimidazo[1,2-a]pyridin-5(1H)-one (18.2 g) was treated with 1,1-dimethylethyl 4-piperidinylcarbamate (26.6 g, 132.8 mmole) in acetonitrile (900 ml) then pyridine (10.7 ml, 132 mmol). The mixture was heated at 60° C. under argon for 17 hours and then heated at 70° C. for 2 hours, cooled and evaporated to about half the volume. The thick yellow precipitate was removed by filtration and washed well with diethyl ether. The filtrate was evaporated to dryness and the residue partitioned between chloroform (500 ml) and water (200 ml). The undissolved material was removed by filtration and washed with chloroform (100 ml). The layers in the filtrate were separated and the aqueous layer was washed with chloroform (200 ml). The combined organic extracts were passed through a hydrophobic frit and evaporated to a dark yellow gum which was chromatographed eluting with 0 to 100% ethyl acetate in hexane then 0 to 30% methanol in ethyl acetate to give a yellow solid (10.98 g).

C₁₈H₂₇N₅O₅ requires 393, MS (ES+) m/z 394(MH⁺).
(d) 1,1-Dimethylethyl {1-[(3,8-dioxo-1,2,5a,8b-tetrahydro-3H,8H-2a,5,8a-triazaacenaphthylen-1-yl)methyl]-4-piperidinyl}carbonate

A suspension of 1,1 -dimethylethyl {1-[(8-nitro-5-oxo-1,2,3,5-tetrahydroimidazo[1,2-a]pyridin-3-yl)methyl]-4-piperidinyl}carbamate (2.0 g, 5.08 mmol) and anhydrous potassium carbonate (700 mg, 5.06 mmol) in absolute alcohol (150 ml) was hydrogenated at atmospheric pressure in the presence of 10% Pd on C (1 g) for 4 hours. The reaction was filtered through silica gel, washed through with ethanol (100 ml) and the dark purple mixture was reacted immediately by treating with anhydrous potassium carbonate (1.4 g, 10 mmol) and ethyl bromoacetate (550 ul, 4.95 mmol) and stirred at room temperature for 20 hours and then heated at 60° C. for 30 minutes. After 45 minutes a further 0.25 ml of ethyl bromacetate was added and heated at 60° C. for 1.5 hours. 0.25 ml of ethyl bromacetate was added and the reaction was again heated at 60° C. for 1 hour. The reaction was filtered through Keiselguhr and evaporated to dryness. The mixture was azeotroped with chloroform and then chromatographed eluting with 0 to 100% ethyl acetate in hexane and then with 0 to 20% methanol in ethyl acetate. A second purification eluting with 0 to 50% methanol in ethyl acetate gave a dark gum (37 mg, 1.6%).

C₂₀H₂₇N₅O₂ requires 401, MS (ES+) m/z 402 (MH⁺).

(e) Title compound: 1-[(4-Amino-1-piperidinyl)methyl]-1,2,5a,8b-tetrahydro-3H,8H-2a,5,8a-triazaacenaphthylene-3,8-dione A solution of 1,1-dimethylethyl {1-[(3,8-dioxo-1,2,5a,8b-tetrahydro-3H,8H-2a,5,8a-triazaacenaphthylen-1-yl)methyl]-4-piperidinyl}carbamate (37 mg, 0.092 mmol) in anhydrous dichloromethane (2 ml) was treated with TFA (1 ml) and stirred at room temperature for 1 hour, evaporated to dryness, mixed with anhydrous dichloromethane and evaporated to a dark gum. This gum was dissolved in 1:1 dichloromethane:methanol (10 ml) and treated with MP-carbonate resin (600 mg) and stirred for 1.5 hours. The reaction was filtered and the resin was washed with 1:1 dichloromethane:methanol (30 ml) and the filtrate was evaporated to dryness. Purification on a 5 g SCX column eluting with a methanol to 2N methanolic ammonia gradient gave the product as a gum. Further evaporation from diethyl ether gave the product as a brown solid (22.8 mg, 82%).

C₁₅H₁₉N₅O₂ requires 301, MS (ES+) m/z 302 (MH⁺).

### Preparation 4

(2E)-3-(2,5-Difluorophenyl)-2-propenal (Preparitive Scheme 4).

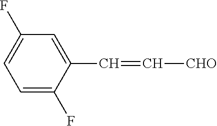

(a) Methyl (2E)-3-(2,5-difluorophenyl)-2-propenoate.

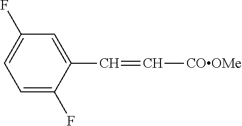

To a suspension of trans-2,5-difluorocinnamic acid in methanol at 0° C. was added thionyl chloride and the mixture was stirred at room temperature overnight. The reaction mixture was evaporated and the product purified on silica gel column and eluted with hexane/ethyl acetate gradient 0-80% to give 297 mg of white solid.

(b) (2E)-3-(2,5-Difluorophenyl)-2-propen-1-ol

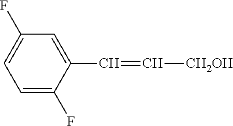

To a solution of methyl (2E)-3-(2,5-difluorophenyl)-2-propenoate in DCM (6 ml) at −78° C. under N₂ was added DIBAL-H 1.5M toluene (2.21 ml, 3.22 mmol). The mixture was stirred at −78° C. for 30 min. Further DIBAL-H 1.5M (0.3 ml) was added and after 10 min at the same temperature, the reaction was completed. A mixture of methanol/acetic acid (2 ml/1 ml) and water was added to the reaction mixture. The layers were separated and the organic layer was washed with saturated NaCl, dried over Na₂SO₄, filtered and evaporated under reduced pressure to give 116 mg of desired product.

### (c) Title Compound

To a solution of (2E)-3-(2,5-difluorophenyl)-2-propen-1-ol (116 mg, 0.68 mmol) was added MnO₂ (474 mg, 5.45 mmol) and the mixture was stirred at room temperature overnight. The reaction mixture was filtered and it was evaporated to give 93 mg of white solid.

### Preparation 5

(2E)-3-(3-Fluorophenyl)-2-propenal (Preparitive Scheme 4).

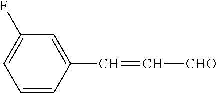

(a) Methyl (2E)-3-(3-Fluorophenyl)-2-propenoate.

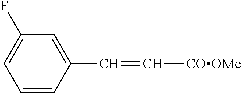

To a suspension of (2E)-3-(3-fluorophenyl)-2-propenoic acid (840 mg, 1 eq) in methanol (8 ml) at 0° C. was added thionyl chloride (0.443 ml, 1.2 eq) dropwise. The reaction mixture was stirred at rt overnight. The reaction mixture was concentrated under reduced pressure to give 904 mg of product as a yellow liquid.

### MS (ES+) m/z 181 (MH+).

(b) (2E)-3-(3-Fluorophenyl)-2-propen-1-ol.

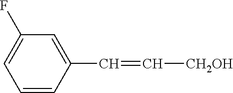

To a solution of methyl (2E)-3-(3-fluorophenyl)-2-propenoate in DCM (15 ml) stirred under nitrogen at 0° C. was added a solution of DIBAL-H 1.5 M in toluene dropwise. The reaction mixture was stirred at 0° C. for 3 hour. A solution of Na,K Tartrate saturated in water was added, and then it was extracted 3 times with DCM. The combined organic phases were washed with saturated NaCl, dried over anhydrous Na₂SO₄ and concentrated to give 711 mg of title compound as a colorless liquid that was used in next step without further purification.

### (c) Title Compound.

Manganese dioxide (2.844 g, 32.7 mmol) was added to a solution of (2E)-3-(3-fluorophenyl)-2-propen-1-ol (711 mg, 4.67 mmol) in DCM (30 ml) at rt and the mixture was stirred at that temperature overnight. More manganese dioxide (2.844 g, 32.7 mmol) was added until TLC showed full conversion. The solids were filtered off and the solvent evaporated, yielding title compound (587.7 mg, 3.91 mmol, 84% yield) N4890-39-1 as a pale yellow oil.

### MS (ES+) m/z 151 (MH+).

### Preparation 6

3-(3-Fluorophenyl) propanal (Preparitive Scheme 5)

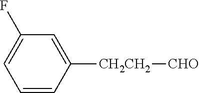

(a) Methyl 3-(3-fluorophenyl)propanoate

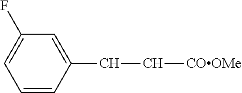

To a suspension of 3-(3-fluorophenyl)propanoic acid (840 mg, 5.00 mmol) in methanol (8 ml) at 0° C. was added thionyl chloride (0.443 ml, 5.99 mmol) dropwise. The reaction mixture was stirred at room temperature overnight. The reaction mixture was concentrated under reduced pressure to give 904 mg (94%) of title compound as a colorless liquid that was used in next step without further purification.

¹H-NMR (δ, ppm, CDCl₃): 7.28-7.21 (m, 1H), 6.99-6.87 (m, 3H), 3.68 (s, 3H), 2.95 (t, 2H), 2.64 (t, 2H).

(a) 3-(3-Fluorophenyl)-1-propanol

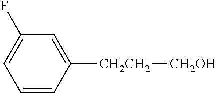

To a solution of methyl 3-(3-fluorophenyl)propanoate (905 mg, 4.97 mmol) in DCM (15 ml) stirred under nitrogen at 0° C. was added a solution of DIBAL-H in toluene 1.5 M dropwise (7.12 ml, 10.68 mmol). The reaction mixture was stirred at −78° C. for 3 h. A NaK-tartrate saturated solution in water was added dropwise, and then it was extracted 3 times with DCM. The combined organic phases were washed with saturated NaCl, dried over anhydrous Na₂SO₄ and concentrated to give 626.4 mg (82%) of product that was used in next step without further purification.

¹H-NMR (δ, ppm, CDCl₃): 7.28-7.20 (m, 1H), 6.99-6.85 (m, 3H), 3.68 (t, 2H), 2.72 (t, 2H), 1.94-1.84 (m, 2H), 1.31 (bs, 1H).

### (b) Title Compound

Dimethyl sulfoxide (333 μl, 4.69 mmol) was added to a solution of (ClCO)₂ (1.004 ml, 2.008 mmol) in DCM (7 ml) at −78° C. After 5 min, a solution of 3-(3-fluorophenyl)-1-propanol (206.5 mg, 1.339 mmol) in DCM (4 ml) was added dropwise and stirred for 15 min at −78° C. Triethylamine (784 μl, 5.62 mmol) was slowly added and the mixture was stirred at −78° C. for 10 min and then allowed to attain rt and stirred for an additional hour. Water and t-BuOMe were added. Extraction, drying (MgSO₄), filtration, and concentration afforded 242.1 mg of crude product. Purification by chromatography using a 5 g silica gel cartridge, and mixtures of DCM and hexane as eluent afforded 135.4 mg (66%) of title compound.

¹H-NMR (δ, ppm, CDCl₃): 9.80 (s, 1H), 735.-7.20 (m, 1H), 7.05-6.85 (m, 3H), 3.05-2.90 (m, 2H), 2.85-2.60 (m, 2H).

### Preparation 7

(2E)-3-(5-Fluoro-3-pyridinyl)-2-propenal (Preparitive Scheme 6)

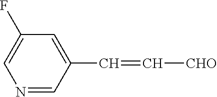

A mixture of 3-bromo-5-fluoropyridine (816 mg, 4.64 mmol), trans-DI(μ-acetato)bis[o-(di-o-tolyl-phosphino)benzyl]dipalladium(II) (87 mg, 0.093 mmol) and sodium acetate (423 mg, 5.16 mmol) was introduced in a pressure tube under argon. Then NMP (12 ml) previously degassed and then 2-propenal (684 μl, 4.64 mmol) was consecutively added to the reaction mixture. After 2 h at 140° C. argon atmosphere the reaction was completed. H₂O and DCM was added and the organic layer was separated, washed with brine, dried over Na₂SO₄ and solvent removed by vacuum. NMP was removed under vacuum to give 425 mg of a brown oil. The crude product was purified by silica chromatography (10 g Si, Hexane:ethyl acetate 8:2) to give 87 mg of a pale solid.

### MS (ES+) m/z 152 [M+H]⁺

### Preparation 8

[(3-Fluorophenyl)oxy]acetaldehyde (Preparitive Scheme 7)

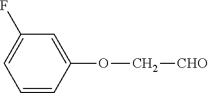

(a) Methyl [(3-fluorophenyl)oxy]acetate.

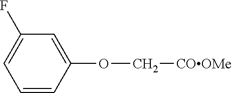

To a solution of [(3-fluorophenyl)oxy]acetic acid (200 mg, 1.175 mmol) in methanol (3 ml) was added thionyl chloride (0.086 ml, 1.175 mmol) and the mixture was stirred at room temperature for 3 h. The reaction mixture was evaporated to dryness to give 203 mg of colorless oil.

### (b) Title Compound

To a solution of methyl [(3-fluorophenyl)oxy]acetate (200 mg, 1.086 mmol) in DCM (6 ml) at −78° C. under N₂ was added a solution of DIBAL-H (1.140 ml of 1M solution in THF, 1.140 mmol). The mixture was stirred at −78° C. After 1 h at −78° C. 0.76 mL of DIBAL-H (1.5 M solution in toluene, 1.140 mmol) were added to the reaction mixture and then stirring was continued at −78° C. for 1.5 h. As the conversion of the reaction was not complete 0.760 ml DIBAL-H (1.5 M solution in toluene 1.140 mmol) was added and the mixture finally stirred for an addition 1 h at −78° C. The reaction mixture was diluted with DCM, Rochelle salt (potassium sodium tartrate tetrahydrate) solution in water was added. The layers were separated and the organic layer was dried over Na₂SO₄, filtered and evaporated under reduced pressure to give 144 mg of colorless oil.

### Preparation 9

2-[(3-Fluorophenyl)amino]ethyl methanesulfonate (Preparitive Scheme 8).

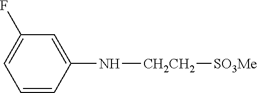

(a) 2-[(3-Fluorophenyl)amino]ethyl chloridocarbonate.

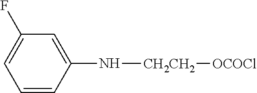

To a solution of 3-fluoroaniline (1 g, 9.00 mmol) in DCM (16 ml) and pyridine anhydrous (0.917 ml, 11.34 mmol) ,2-chloroethyl chloroformate (0.930 ml, 9.00 mmol) was added at 0° C. under N₂. The reaction mixture was stirred at room temperature for 20 h. H₂O was added and organic layer was extracted, dried over Na₂SO₄, filtered and evaporated under reduced pressure to give 1.82 g of brown oil that was used in the next step without further purification.

(b) 2-[(3-Fluorophenyl)amino]ethanol.

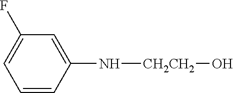

To a solution of 2-[(3-fluorophenyl)amino]ethyl chloridocarbonate (1.82 g, 8.36 mmol) in EtOH (12 ml) , KOH (1.88 g, 33.5 mmol) was added. The mixture was heated at 90° C. for 2 h under N₂. The reaction mixture was evaporated to dryness and the residue was washed with NaCl sat solution and extracted with DCM. Organic layer was dried over MgSO₄, filtered and concentrated under reduced pressure to give 1.20 g of brown liquid that was used in the next step without further purification.

### (c) Title Compound

To a solution of 2-[(3-fluorophenyl)amino]ethanol (1.20 g, 7.73 mmol) in DCM (12 ml), Et₃N (1.3 ml, 9.28 mmol) was added under N₂. The reaction mixture was stirred for 20 min at room temperature, then it was cooled at 0° C. and methanesulfonyl chloride (0.66 ml, 8.51 mmol) was added. The suspension was stirred at room temperature for 20 h. H₂O was added and organic layer was separated, washed with NaCl sat solution, dried over MgSO₄, filtered and concentrated under reduced pressure to give 1.70 g of title compound as brown liquid.

### MS (ES+) m/z 234 [M+H]⁺.

### Preparation 10

(2E)-3-[5-bromo-2-(methyloxy)-3-pyridinyl]-2-propenal

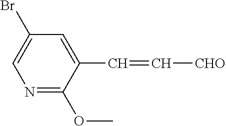

(a) (2E)-3-[5-bromo-2-(methyloxy)-3-pyridinyl]-2-propen-1-ol

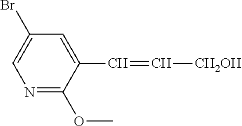

To a solution of ethyl 3-(5-bromo-2-methoxypyridin-3-yl)acrylate (180 mg, 0.629 mmol) in DCM (4 ml) stirred under nitrogen at 0° C. was added a solution of DIBAL-H 1.5 M in toluene dropwise (0.902 ml, 1.353 mmol). The reaction mixture was stirred at 0 ° C. for 3 h. The reaction mixture was diluted with MeOH dropwise and concentrated under reduced pressure. The residue was treated with a 1N HCl solution and extracted 3 times with EtOAc. The combined organic phases were washed with saturated NaCl, dried over anhydrous Na₂SO₄ and concentrated to give 174 mg of title compound that was used in next step without further purification.

### (c) Title Compound.

Manganese dioxide (427 mg, 4.92 mmol) was added to a solution of (2E)-[5-bromo-2-(methyloxy)-3-pyridinyl]-2-propen-1-ol (150 mg, 0.615 mmol) in DCM (4 ml) at rt and the mixture was stirred at that temperature overnight. More manganese dioxide (214 mg, 2.458 mmol) was added until TLC showed full conversion. The solids were filtered off and the solvent evaporated and the residue was purified by Flash-Master chromatography on a 2 g silica gel cartridge using 50% Hex/EtOAc as eluent. The same cartridge was eluted with DCM/MeOH gradient 0-10% to yield the title compound (90 mg, 60% yield) as a pale yellow solid.

### MS (ES+) m/z 242 (MH⁺).

### Preparation 11

3-(3-Fluorophenyl)-2-propynal

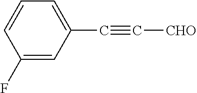

(a) 3-(3-Fluorophenyl)-2-propyn-1-ol

A mixture of copper(I) iodide (0.017 g, 0.090 mmol) and bis(triphenylphosphine)palladium(II) chloride (0.032 g, 0.045 mmol) in diisopropylamine (15 ml) and triethylamine (5 ml) was stirred under N₂ and 1-fluoro-3-iodobenzene (1 g, 4.50 mmol) was added and stirred for 30 min. Then, 2-propyn-1-ol (0.278 g, 4.95 mmol) was added and the reaction mixture was heated at 60° C. for 90 min. Then it was allowed to attain the room temperature and stirred for 90 min. The crude reaction was filtrated and the solid was washed with DCM, then the solvent was evaporated giving 1.30 g of a brown solid. The crude reaction was purified by flash cromatography using FlashMaster II, gel silica cartridge and mixtures of EtOAc/Hexane as eluents to afford the desired product 3-(3-fluorophenyl)-2-propyn-1-ol (638 mg, 94%).

¹H-NMR (δ, ppm, CDCl₃): 7.35-6.95 (m, 4H), 4.50 (s, 2H).

### (b) Title Compound

3-(3-Fluorophenyl)-2-propyn-1-ol (200 mg, 1.332 mmol) was dissolved in DCM (10 ml) and CrO₂ Magtrieve™ (1678 mg, 19.98 mmol) was added. The reaction mixture was stirred at 40° C. overnight. The reaction mixture was filtrated and evaporated to afford 125 mg of 3-(3-fluorophenyl)-2-propynal (125 mg, 63.4%) pure enough to be used it in the next step.

¹H-NMR (δ, ppm, CDCl₃): 9.30 (s, 1H), 7.30-7.00 (m, 4H).

### Preparation 12

(2Z)-3-(3-Fluorophenyl)-2-propenal

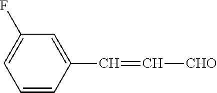

(a) (2Z)-3-(3-Fluorophenyl)-2-propen-1-ol

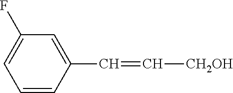

To a solution of 3-(3-fluorophenyl)-2-propyn-1-ol (100 mg, 0.666 mmol) in toluene (6 ml) were added quinoline (0.039 ml, 0.333 mmol) and Pd on CaCO₃, poisoned with lead (Lindlar catalyst) (31.6 mg, 0.153 mmol). The mixture was hydrogenated at 29 psi. After 1.5 h, the reaction mixture was filtered and it was purified on silica gel column and was eluted with DCM/EtOAc gradient 0-20%. Collected fractions were evaporated under reduced pressure to give 29 mg of impure product and 36 mg of pure product as a mixture Z/E 9:1.

¹H-NMR (δ, ppm, CDCl₃): 7.34-7.22 (m, 1H), 7.01-6.91 (m, 3H), 6.53 (d, 1H), 5.96-5.88 (m, 1H), 4.43 (dd, 2H).

### (b) Title Compound

To a solution of (2Z)-3-(3-fluorophenyl)-2-propen-1-ol (Z/E 9:1) (37 mg, 0.243 mmol) in Dichloromethane (DCM) (3 ml) was added Manganese (IV) oxide (169 mg, 1.945 mmol) and the mixture was stirred at room temperature for 7 h. TLC (DCM/MeOH 9:1) showed the reaction was complete.

The reaction mixture was filtered through celite and it was evaporated to dryness to give 35 mg of the title compound as yelow oil.

¹H-NMR (δ, ppm, CDCl₃): 9.96 (d, 1H), 7.57 (d, 1H), 7.44-7.37 (m, 1H), 7.19-7.09 (m, 3H), 6.22-6.19 (m, 1H).

### Preparation 13

2-(3,4-Dichlorophenyl)ethyl methanesulfonate

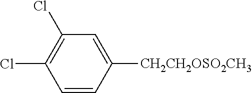

2-(3,4-Dichlorophenyl)ethanol (150 mg, 0.769 mmol) was dissolved in (DCM (3 mL) and when the temperature was cooled down at 0° C. methanesulfonyl chloride (0.066 mL, 0.846 mmol) was slowly added leaving the mixture under stirring overnight. Next morning HPLC showed completion. Volatiles were removed under vacuum and the mixture was treated with water and EtOAc. Organic layers were dried over Na₂SO₄, filtered and the solvent removed under vacuum to obtain 200 mg of the desired compound as colourless oil that was used in the next step without further purification.

The compounds of Formula IIA and IIB were used to prepare Example compounds, of which Examples 1 and 2 listed below are representative.

### Example 1

(1R)-1-[(4-{[(2E)-3-(2,5-difluorophenyl)-2-propen-1-yl]amino}-1-piperidinyl)methyl]-1,2-dihydro-4H,9H-imidazo[1,2,3-ij]-1,8-naphthyridine-4,9-dione.

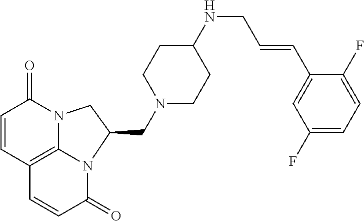

To a solution of (1R)-1-[(4-amino-1-piperidinyl)methyl]-1,2-dihydro-4H,9H-imidazo[1,2,3-ij]-1,8-naphthyridine-4,9-dione (15 mg, 0.050 mmol) (Preparation 1 above) in a mixture of DCM (0.5 ml) and methanol (0.050 ml), MgSO₄ (15 mg, 0.125 mmol) and (2E)-3-(2,5-difluorophenyl)-2-propenal (8.40 mg, 0.050 mmol) (see Preparation 4 above) were added and the mixture was stirred at room temperature for 90 minutes. NaBH(OAc)₃ (63.5 mg, 0.300 mmol) and more DCM (0.5 ml) were then added and the mixture was stirred at room temperature for 2.5 h. 10% NaHCO₃ aqueous solution and DCM were added. The organic layer was extracted, dried (MgSO₄), filtered and concentrated, yielding 27.6 mg of crude material. Purification by flash chromatography using Flashmaster II, a 500 mg silica gel cartridge, and mixtures of DCM and methanol as an eluant afforded title compound but not pure at all. The resulting solid was triturated with hexane and isolated by filtration to provide the free base of the title compound as a pale yellow solid (14 mg, 62%).

¹H-NMR (δ, ppm, CDCl₃): 7.50 (d, 1H); 7.49 (d, 1H); 7.17-7.08 (m, 1H); 7.03-6.93 (m, 1H); 6.92-6.82 (m, 1H); 6.64 (d, 1H); 6.35 (dt, 1H); 6.31 (d,1H); 6.26 (d,1H); 5.07-4.96 (m, 1H); 4.58 (dd, 1H); 4.37 (dd, 1H); 3.45 (dd, 2H); 3.10 (dd, 1H); 3.02-2.92 (m, 1H); 2.70-2.60 (m, 1H); 2.69 (dd, 2H); 2.58-2.46 (m, 1H); 2.38-2.19 (m, 2H); 1.94-1.76 (m, 2H); 1.42-1.21 (m, 2H).

### MS (ES+) m/z 152 [M+H]⁺

### Example 2

2-[(4-{[(2E)-3-(3-fluorophenyl)-2-propen-1-yl]amino}-1-piperidinyl)methyl]-1,2-dihydro-3H,8H-2a,5,8a-triazaacenaphthylene-3,8-dione.

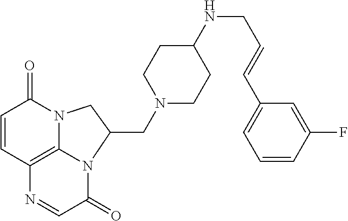

A suspension of 2-[(4-Amino-1-piperidinyl)methyl]-1,2-dihydro-3H,8H-2a,5,8a-triazaacenaphthylene-3,8-dione (Preparation 2 above) (80 mg, 0.265 mmol), magnesium sulphate (80 mg, 0.665 mmol), and (2E)-3-(3-fluorophenyl)-2-propenal (See Preparation 5 above) (39.9 mg, 0.265 mmol) in a mixture of Dichloromethane (DCM) (4 ml) and Methanol (0.6 ml) was stirred at room temperature for 100 min. NaBH(OAc)3 (394 mg, 1.858 mmol) was then added and the mixture stirred at room temperature overnight. Next morning, full conversion was observed by LCMS. It was quenched with 10% NaHCO3 and extracted with dichloromethane. The organic layer was dried (MgSO4), filtered, and concentrated. Purification by flash chromatography (pre-loading of the sample on silica was necessary) using Flashmaster II, a 2 g silica gel cartridge, and mixtures of dichloromethane and methanol as an eluant afforded 2-[(4-{[(2E)-3-(3-fluorophenyl)-2-propen-1-yl]amino}-1-piperidinyl)methyl]-1,2-dihydro-3H,8H-2a,5,8a-triazaacenaphthylene-3,8-dione (46.7 mg, 0.107 mmol, 40.4% yield) N4890-41-1 as a yellow solid.

1H-NMR (d, ppm, CDCl3): 7.83 (s,1H); 7.78 (d,1H); 7.30-7.23 (m,1H); 7.15-7.02 (m,2H); 6.92 (dt,1H); 6.50 (d,1H); 6.40 (d,1H); 6.30 (dt,1H); 5.09-4.99 (m,1H); 4.56 (dd,1H); 4.39 (dd,1H); 3.43 (dd,2H); 3.15 (dd,1H); 3.00-2.90 (m,1H); 2.74-2.63 (m,1H); 2.72 (dd,1H); 2.60-2.48 (m,1H); 2.41-2.21 (m,2H); 1.94-1.79 (m,2H); 1.38-1.23 (m,2H).

### Example 3

2-[(4-{[3-(3-fluorophenyl)propyl]amino}-1-piperidinyl)methyl]-1,2-dihydro-3H,8H-2a,5,8a-triazaacenaphthylene-3,8-dione

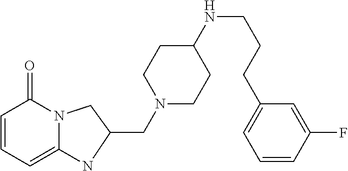

A suspension of 2-[(4-Amino-1-piperidinyl)methyl]-1,2-dihydro-3H,8H-2a,5,8a-triazaacenaphthylene-3,8-dione (Preparation 2 above) (36.8 mg, 0.122 mmol), magnesium sulphate (37 mg, 0.307 mmol), and 3-(3-fluorophenyl)propanal (see Preparation 6 above) (18.6 mg, 0.122 mmol) in a mixture of Dichloromethane (DCM) (2 ml) and Methanol (0.2 ml) was stirred at room temperature for 2 h. NaBH(OAc)3 (181 mg, 0.856 mmol) was then added and the mixture stirred at room temperature overnight. Next morning, full conversion was observed by LCMS. It was quenched with 10% NaHCO3 and extracted with dichloromethane. The organic layer was dried (MgSO4), filtered, and concentrated, affording 38.5 mg of crude material. Purification by flash chromatography using a 1 g silica gel cartridge, and mixtures of dichloromethane and methanol as an eluant afforded 2-[(4-{[3-(3-fluorophenyl)propyl]amino}-1-piperidinyl)methyl]-1,2-dihydro-3H,8H-2a,5,8a-triazaacenaphthylene-3,8-dione (23.5 mg, 0.054 mmol, 43.9% yield) N4890-43-1 as a greenish yellow solid.

1H-NMR (d, ppm, CDCl3): 7.83 (s,1H); 7.77 (d,1H); 7.27-7.20 (m,1H);6.99-6.84 (m,3H); 6.39 (d,1H); 5.07-4.99 (m,1H); 4.55 (dd,1H); 4.38 (dd,1H); 3.13 (dd,1H); 2.98-2.88 (m,1H); 2.74-2.61 (m,6H); 2.51-2.39 (m,1H); 2.37-2.20 (m,2H); 1.89-1.74 (m,2H); 1.37-1.22 (m,2H).

### Example 4

(1R)-1-[(4-{[(2E)-3-(5-fluoro-3-pyridinyl)-2-propen-1-yl]amino}-1-piperidinyl)methyl]-1,2-dihydro-4H,9H-imidazo[1,2,3-ij]-1,8-naphthyridine-4,9-dione.

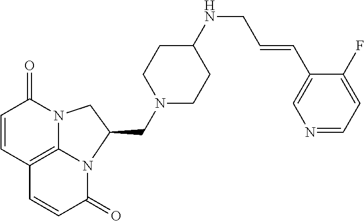

To a solution of (1R)-1-[-(4-Amino-1-piperidinyl)methyl]-1,2-dihydro-4H,9H-imidazo[1,2,3-ij]-1,8-naphthyridine-4,9-dione dihydrochloride. (Preparation 1 above) DCM and MeOH (a few drops until dissolution of mixture) was added (2E)-3-(5-fluoro-3-pyridinyl)-2-propenal (see Preparation 7 above) and a spatule of MgSO4 .The mixture was stirred at room temperature for 16 h under N₂. LCMS shows the imine, then sodium triacetoxyborohydride was added. The mixture was stirred at room temperature for 3 h under N₂. LCMS of the crude reaction product showed reaction had gone to completion. MgSO4 was filtered off, solvent was evaporated in vacuo and the obtained solid was dissolved in DCM.The organic phase was washed with sat NaHCO₃, dried over Na₂SO₄, filtered and evaporated under reduced pressure to give 53.3 mg of a brown oil. The crude was purified by flash master (2 gr Si, 9:1 DCM/MeOH) to give 17 mg of a pink solid N5126-41-1. HNMR:N5126-41-1 H45266a was consistent with desired compound .LCMS:N5126-41-1 peak at RT=1.67 was the desired compound plus some starting reagents.

1H-NMR (d, ppm, CDCl3): 8.39 (bs,1H); 8.31 (d,1H); 7.49 (dd,2H); 7.39 (dt,1H); 6.56-6.50 (m, 1H); 6.43-6.34 (m,1H); 6.27 (dd,2H); 5.05-4.97 (m,1H); 4.57 (dd,1H); 4.35 (dd,1H); 3.46 (dd,2H); 3.10 (dd,1H); 3.01-2.93 (m,1H); 2.68 (dd,2H); 2.58-2.46 (m,1H); 2.38-2.20 (m,2H); 1.91-1.81 (m,2H); 1.36-1.28 (m,2H).

### Example 5

2-[(4-{[(2E)-3-(5-fluoro-3-pyridinyl)-2-propen-1-yl]amino}-1-piperidinyl)methyl]-1,2-dihydro-3H,8H-2a,5,8a-triazaacenaphthylene-3,8-dione.

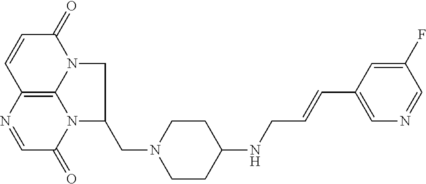

A mixture of (2E)-3-(5-fluoro-3-pyridinyl)-2-propenal (see Preparation 7 above) (40 mg, 0.265 mmol), 2-[(4-amino-1-piperidinyl)methyl]-1,2-dihydro-3H,8H-2a,5,8a-triazaacenaphthylene-3,8-dione (Preparation 2 above) (80 mg, 0.265 mmol) and magnesium sulphate (60 mg, 0.498 mmol) in DCM and MeOH was stirred at room temperature for 3 h. Then sodium triacetoxyborohydride (337 mg, 1.588 mmol) was added and the reaction was stirred over night. LCMS showed some imine left and more sodium triacetoxyborohydride (57 mg, 0.269 mmol) was added. After 2 hours LCMS showed completion. Then NaHCO₃ sat. and DCM were added, the organic phase was separated, dried over Na₂SO₄, filtered and concentrated to give 136 mg. Purification by flash chromatography using FlashMaster II and mixtures of dichloromethane and methanol as an eluant afforded 2-[(4-{[(2E)-3-(5-fluoro-3-pyridinyl)-2-propen-1-yl]-amino}-1-piperidinyl)methyl]-1,2-dihydro-3H,8H-2a,5,8a-triazaacenaphthylene-3,8-dione (40 mg, 0.092 mmol, 34.6% yield) N5007-52-1 as a yellow solid. (See HNMR and LCMS).

1H-NMR (d, ppm, CDCl3): 8.40 (bs,1H); 8.33 (d,1H); 7.84 (s,1H); 7.40 (dt,1H); 6.54 (d,1H); 6.44-6.34 (m,2H); 5.09-4.99 (m,1H); 4.56 (dd,1H); 4.39 (dd,1H); 3.46 (dd,2H); 3.15 (dd,1H); 2.99-2.92 (m,1H); 2.76-2.66 (m,2H); 2.59-2.49 (m,1H); 2.41-2.24 (m,2H); 1.93-1.82 (m,2H); 1.37-1.25 (m,2H).

### Example 6

2-{[4-({2-[(3-fluorophenyl)oxy]ethyl}amino)-1-piperidinyl]methyl}-1,2-dihydro-3H,8H-2a,5,8a-triazaacenaphthylene-3,8-dione.

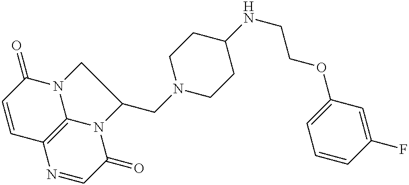

To a solution of 2-[(4-amino-1-piperidinyl)methyl]-1,2-dihydro-3H,8H-2a,5,8a-triazaacenaphthylene-3,8-dione (Preparation 2 above) (30 mg, 0.100 mmol) in Dichloromethane (DCM) (5 ml) and Methanol (0.5 ml) was added anhydrous MgSO4 (30 mg) and [(3-fluorophenyl)oxy]acetaldehyde (see Preparation 8 above) (15.35 mg, 0.100 mmol) and it was stirred at room temperature for 2 h. NaBH(OAc)₃ (127 mg, 0.597 mmol) was added and it was stirred for 2 h. LCMS showed the reaction was complete. LCMS:N4911-73-RM1 The reaction mixture was filtered and it was washed with saturated NaHCO3. The organic layer was dried over Na₂SO₄, filtered and evaporated under reduced pressure. The crude was purified on silica gel column and was eluted with CH₂Cl₂ (R40)/MeOH (R11, R23/24/25, R39/23/24/25) gradient 0-20%. Collected fractions were evaporated under reduced pressure to give 20.5 mg of yelow solid. (N4911-73-1):-Analysis by 1H NMR in CDCl₃ is consistent with desired product. NMR:N4911-73-1-LC-MS analysis showed a purity of >95% (ES+: m/z=440). LCMS:N4911-73-1.

1H-NMR (d, ppm, CDCl3): 7.83 (s,1H); 7.76 (d,1H); 7.22-7.17 (m,1H); 6.70-6.59 (m,3H); 6.38 (d,1H); 5.08-4.99 (m,1H); 4.58-4.52 (m,1H); 4.42-4.34 (m,1H); 4.04 (t,2H); 3.17-3.11 (m,1H); 2.99 (t,2H); 2.95-2.92 (m,1H); 2.75-2.67 (m,2H); 2.56-2.48 (m,1H); 2.39-2.23 (m,2H); 1.90-1.80 (m,2H); 1.37-1.25 (m,2H).

### Example 7

2-{[4-({2-[(3-Fluorophenyl)amino]ethyl}amino)-1-piperidinyl]methyl}-1,2-dihydro-3H,8H-2a,5,8a-triazaacenaphthylene-3,8-dione

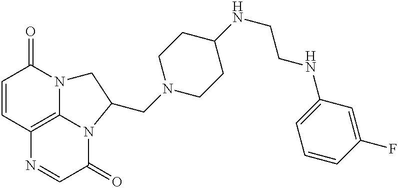

To a solution of 2-[(4-amino-1-piperidinyl)methyl]-1,2-dihydro-3H,8H-2a,5,8a-triazaacenaphthylene-3,8-dione (50.4 mg, 0.167 mmol) (Preparation 2 above) in a mixture of ACN (2 ml) and MeOH (0.3 ml), potassium carbonate (30.8 mg, 0.223 mmol) was added under N₂. The mixture was stirred at room temperature for 10 min then a solution of 2-[(3-fluorophenyl)amino]ethyl methanesulfonate (26 mg, 0.111 mmol) (see Preparation 9 above) in ACN (1 ml) was added. The reaction mixture was stirred at room temperature for 20 h and then at 80° C. for 20 h. The solid was filtered off and the solvent was evaporated under reduced pressure to give 61.17 mg of yellow oil. The crude reaction was purified by Flashmaster II (2 g silica gel cartridge) using a DCM-methanol gradient to provide the free base of the title compound as a pale yellow solid (21.7 mg, 45%).

¹H-NMR (δ, ppm, CDCl₃): 7.83-7.74 (m, 2H); 7.13-7.04 (m, 1H); 6.44-6.29 (m, 4H), 5.08-4.97 (m, 1H); 4.52 (dd, 1H); 4.39 (dd, 1H); 3.29 (bs, 2H); 3.16 (dd, 1H); 3.01-2.93 (m, 3H); 2.75-2.55 (m, 3H); 2.38-2.19 (m, 2H); 1.98-1.83 (m, 2H); 1.54-1.38 (m, 2H).

### [ES MS] m/z 439 (MH+).

### Example 8

(1R)-1-{[4-({2-[(3-fluorophenyl)amino]ethyl}amino)-1-piperidinyl]methyl}-1,2-dihydro-4H,9H-imidazo[1,2,3-ij]-1,8-naphthyridine-4,9-dione.

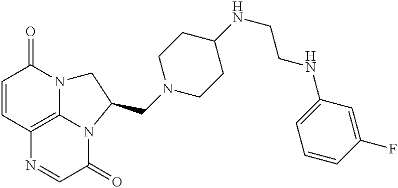

To a solution of (1R)-1-[(4-amino-1-piperidinyl)methyl]-1,2-dihydro-4H,9H-imidazo[1,2,3-ij]-1,8-naphthyridine-4,9-dione (Preparation 1 above) in 2 ml of ACN and 0.3 ml of MeOH, 2 eq of potassium carbonate were added. The mixture was stirred at room temperature for 10 min then a solution of 2-[(3-fluorophenyl)amino]ethyl methanesulfonate (see Preparation 9 above) in 1 ml of ACN was added. The mixture was stirred 20 h at room temperature under N₂. LCMS shows a lot of starting material. The mixture was stirred for 7 h at 80° C. under N₂. LCMS shows a little amount of starting material, the mixture was stirred for 20 h more at room temperature. LCMS:N5126-58-on the crude product of the reaction showed the reaction has gone to completion. Peak at RT=2.14 is desired compound. The mixture was filtered to remove the potassium carbonate and then solvent was removed in vacuo to give 89.4 mg of a yellow oil that was purified by flash master (2 g Si, DCM/MeOH 9:1) to give two fractions: 23.2 mg mg of a yellow oil N5126-56-1 HNMR:N5126-56-1 H45642a was consistent with desired compound. LCMS:N5126-56-1 LCMS6140B peak at RT=1.95 was the desired product 438[M+H]+GSK2218518A 12.2 mg of a yellow oil N5126-56-2:HNMR:N5126-56-2 is consistent with impure desired product. LCMS:N5126-56-2 showed desired compound RT=2.14 and a little amount of impurity RT=2.61.

¹H-NMR (δ, ppm, CDCl₃): 7.49 (dd,2H); 7.08 (q,1H); 6.44-6.21 (m,5H), 5.04-4.94 (m,1H); 4.54 (dd,1H); 4.35 (dd,1H); 3.30 (bs,2H); 3.10 (dd,1H); 3.02-2.91 (m,3H); 2.73-2.58 (m,3H); 2.36-2.16 (m,2H); 1.99-1.83 (m,2H); 1.49-1.37 (m,2H).

The compounds of Examples 9-13 below can be prepared by analogous methods to those of Examples 1 to 8 above.

### Example 9

2-[(4-{[(2E)-3-(2-furanyl)-2-propen-1-yl]amino}-1-piperidinyl)methyl]-1,2-dihydro-3H,8H-2a,5,8a-triazaacenaphthylene-3,8-dione.

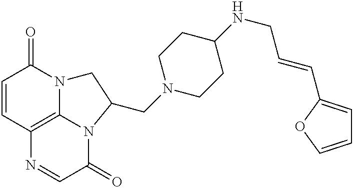

### Example 10

2-{[4-({(2E)-3-[5-bromo-2-(methyloxy)-3-pyridinyl]-2-propen-1-yl}amino)-1-piperidinyl]methyl}-1,2-dihydro-3H,8H-2a,5,8a-triazaacenaphthylene-3,8-dione.

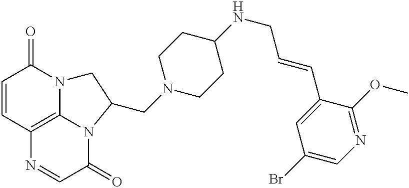

### Example 11

2-[(4-{[(2E)-3-(2-nitrophenyl)-2-propen-1-yl]amino}-1-piperidinyl)methyl]-1,2-dihydro-3H,8H-2a,5,8a-triazaacenaphthylene-3,8-dione.

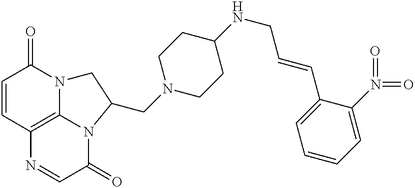

### Example 12

2-[(4-{[(2E)-3-phenyl-2-propen-1-yl]amino}-1-piperidinyl)methyl]-1,2-dihydro-3H,8H-2a,5,8a-triazaacenaphthylene-3,8-dione.

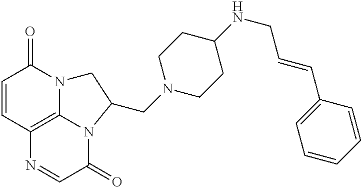

### Example 13

(2E)-3-(2,5-Difluorophenyl)-N-{1-[(3,8-dioxo-1,2-dihydro-3H,8H-2a,5,8a-triazaacenaphthylen-2-yl)methyl]-4-piperidinyl}-2-propenamide

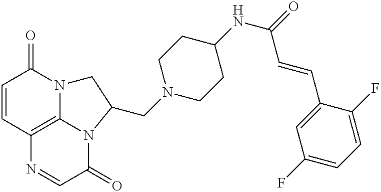

1-Hydroxy-1H-benzotriazol hydrate (30.5 mg, 0.199 mmol) and N-(3-dimethylaminopropyl)-n′-ethylcarbodiimide hydrochloride (38.2 mg, 0.199 mmol) were added to a solution of 2-[(4-amino-1-piperidinyl)methyl]-1,2-dihydro-3H,8H-2a,5,8a-triazaacenaphthylene-3,8-dione (Preparation 2: 50 mg, 0.166 mmol) and trans-2,5-difluorocinnamic acid (33.6 mg, 0.183 mmol) in DCM (4 mL) and DMF (1 mL). The brown supension was stirred at room temperature overnight and monitored by HPLC. The reaction mixture was diluted with DCM and washed with water. The aqueous layer was backextracted with DCM and the combined organic layers were dried over Na₂SO₄, filtared and concentrated. The crude reaction was added to a silica gel column and was eluted with MeOH/DCM mixture to afford desired compound (37 mg) as a yellow solid. HNMR showed an impurity. Solid was dissolved in DCM and washed with sat NaHCO₃. The organic phase was dried over Na₂SO₄, filtered and concentrated to yield desired compound (16 mg, 21%) as a yellow solid.

¹H-NMR (δ, ppm, DMSO-d₆): 8.16 (d, 1H), 7.85 (d, 1H), 7.73 (s, 1H), 7.52-7.46 (m, 1H), 7.40 (d, 1H), 7.36-7.26 (m, 2H), 6.73 (d, 1H), 6.25 (d, 1H), 5.09-4.99 (m, 1H), 4.35-4.21 (m, 2H), 3.67-3.62 (m, 1H), 3.00-2.90 (m, 2H), 2.81 (dd, 1H), 2.65-2.58 (m, 1H), 2.28 (t, 1H), 2.16 (t, 1H), 1.76-1.68 (m, 2H), 1.38-1.27 (m, 2H).

### [ES MS] m/z 468 (MH⁺).

### Example 14

2-[(4-{[(3-Fluorophenyl)ethynyl]amino}-1-piperidinyl)methyl]-1,2-dihydro-3H-8H-2a,5,8a-triazaacenaphthylene-3,8-dione

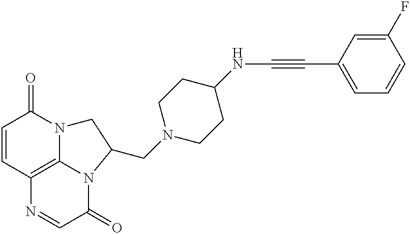

### Example 15

2-[(4-{[(2Z)-3-(3-Fluorophenyl)-2-propen-1-yl]amino}-1-piperidinyl)methyl]-1,2-dihydro-3H,8H-2a,5,8a-triazaacenaphthylene-3,8-dione

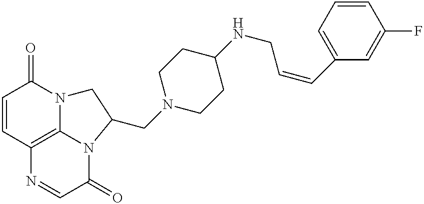

### Example 16

2-[(4-{[2-(3,4-Dichlorophenyl)ethyl]amino}-1-piperidinyl)methyl]-1,2-dihydro-3H-8H-2a,5,8a-triazaacenaphthylene-3,8-dione

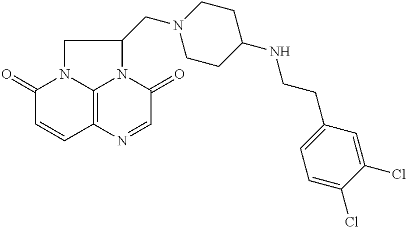

To a solution of 2-(3,4-dichlorophenyl)ethyl methanesulfonate (43.8 mg, 0.163 mmol) (Preparation 13 above) in ACN (5 mL) were added 2-[(4-amino-1-piperidinyl)methyl]-1,2-dihydro-3H,8H-2a,5,8a-triazaacenaphthylene-3,8-dione (Preparation 2: 35 mg, 0.116 mmol) and K₂CO₃ (32.1 mg, 0.232 mmol). The mixture was stirred at 60° C. overnight. HPLC showed still starting material remaining 2 eq more of K₂CO₃ were added and the mixture was left under reflux 3 h. Reaction was very slowly so a small amount of NaI was added in orden to acelerate it, and mixture was left during the weekend at room temperature. HPLC showed still starting material but the reaction was stopped. Volatiles were removed under vacuum and the corresponing crude was diluted with DCM and washed with water several times. Organic layers were dried over Na₂SO₄, filtered and volatiles removed under vacuum. Purification by flash chromatography using a 5 g silica gel cartridge, and mixtures of DCM and MeOH as eluent afforded 4 mg (7%) of the expected compound.

¹H-NMR (δ, ppm, CD₃OD): 7.82 (s, 1H), 7.76 (d, 1H), 7.35 (d, 1H), 7.29 (d, 1H), 7.04 (dd, 1H), 6.37 (d, 1H), 5.08-4.97 (m, 1H), 4.53 (dd, 1H), 4.37 (dd, 1H), 3.13 (dd, 1H), 2.99-2.83 (m, 3H), 2.82-2.75 (m, 4H), 2.57-2.44 (m, 1H), 2.19-2.38 (m, 2H), 1,90-1.71 (m, 2H), 1.41-1.02 (m, 2H).

### [ES MS] m/z 475 (MH⁺).

### Biological Activity

### Antimicrobial Activity Assay:

Whole-cell antimicrobial activity was determined by broth microdilution using the Clinical Laboratory Standards Institute (CLSI) recommended procedure, Document M7-A7, “Methods for Dilution Susceptibility Tests for Bacteria that Grow Aerobically”.

Compounds were evaluated against Gram-positive organisms, including Staphylococcus aureus, Streptococcus pneumoniae, and Enterococcus faecalis.

In addition, compounds were evaluated against Gram-negative organisms including Haemophilus influenzae, Moraxella catarrhalis, and Escherichia coli.

The minimum inhibitory concentration (MIC) was determined as the lowest concentration of compound that inhibited visible growth. A mirror reader was used to assist in determining the MIC endpoint.

Each of the compounds of listed Examples 1, 2, 3, 4 and 6, as identified in Table 1 of the present application, had a MIC<2 μg/ml against at least one of the organisms listed above. For at least one strain of every organism listed above, at least one Example had a MIC<2 μg/ml. The compounds of Examples 5, 7 and 8 were not tested.

Mycobacterium tuberculosis H37Rv Inhibition Assay

The measurement of the minimum inhibitory concentration (MIC) for each tested compound was performed in 96 wells flat-bottom, polystyrene microtiter plates. Ten two-fold drug dilutions in neat DMSO starting at 400 μM were performed. Five μl of these drug solutions were added to 95 μl of Middlebrook 7H9 medium. (Lines A-H, rows 1-10 of the plate layout). Isoniazid was used as a positive control, 8 two-fold dilution of Isoniazid starting at 160 μgml⁻¹ was prepared and 5 μl of this control curve was added to 95 μl of Middlebrook 7H9 (Difco catalogue Ref. 271310)+ADC medium (Becton Dickinson Catalogue Ref 211887). (Row 11, lines A-H). Five μl of neat DMSO were added to row 12 (growth and Blank controls).

The inoculum was standardised to approximately 1×10⁷ cfu/ml and diluted 1 in 100 in Middlebrook 7H9+ADC medium and 0.025% Tween 80 (Sigma P4780), to produce the final inoculum of H37Rv strain (ATCC25618). One hundred μl of this inoculum was added to the entire plate but G-12 and H-12 wells (Blank controls). All plates were placed in a sealed box to prevent drying out of the peripheral wells and they were incubated at 37° C. without shaking for six days. A resazurin solution was prepared by dissolving one tablet of resazurin (Resazurin Tablets for Milk Testing; Ref 330884Y VWR International Ltd) in 30 ml sterile PBS (phosphate buffered saline). 25 μl of this solution was added to each well. Fluorescence was measured (Spectramax M5 Molecular Devices, Excitation 530 nm, Emission 590 nm) after 48 hours to determine the MIC value.

All examples were tested in the Mycobacterium tuberculosis H37Rv inhibition assay. All tested Examples showed an MIC value of 2.4 μg/ml or lower. All tested Examples except Examples 9 and 11 showed an MIC value of 1.0 μg/ml or lower.

TABLE 1

| | | | | | | Starting |
|---|---|---|---|---|---|---|
| | | | | | | compounds of |
| | | | | | Prepn. of | Formula |
| | | Chemical | Physical | MtB MIC | amine | (IIB) and |
| Ex | Structure | name | data/MS | μg/ml | (IIA) | source |
| 1 | 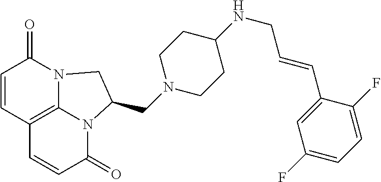 | (1R)-1-[(4-{[(2E)-3- (2,5-difluorophenyl)-2- propen-1-yl]amino}-1- piperidinyl)methyl]- l,2-dihydro-4H,9H- imidazo[l,2,3-ij]-1,8- naphthyridine- 4,9-dione | 1H-NMR(δ, ppm, CDCl₃): 7.50(d, 1H); 7.49(d, 1H); 7.17-7.08 (m, 1H); 7.03-6.93(m, 1H); 6.92-6.82 (m, 1H); 6.64(d, 1H); 6.35(dt, 1H); 6.31(d, 1H); 6.26(d, 1H); 5.07-4.96(m, 1H); 4.58(dd, 1H); 4.37(dd, 1H); 3.45(dd, 2H); 3.10(dd, 1H); 3.02-2.92(m, 1H); 2.70- 2.60(m, 1H); 2.69(dd, 2H); 2.58-2.46(m, 1H); 2.38-2.19(m, 2H); 1.94-1.76(m, 2H); 1.42-1.21(m, 2H). MS: [ES MS] m/z 453(MH+) | 0.071 | Prepn. 1 | (2E)-3-(2,5- difluorophenyl)- 2-propenal Preparation 4 |
| 2 | 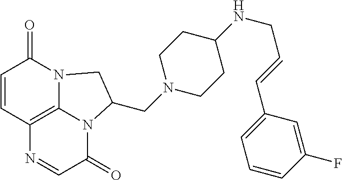 | 2-[(4-{[(2E)-3-(3- fluorophenyl)-2- propen-1-yl]amino}-1- piperidinyl)methyl]- l,2-dihydro-3H,8H- 2a,5,8a- triazaacenaphthylene- 3,8-dione | 1H-NMR(δ, ppm, CDCl₃): 7.83(s, 1H); 7.78(d, 1H);7.30- 7.23(m, 1H); 7.15-7.02(m, 2H); 6.92(dt, 1H); 6.50(d, 1H); 6.40(d, 1H); 6.30(dt, 1H); 5.09-4.99(m, 1H); 4.56(dd, 1H); 4.39(dd, 1H); 3.43(dd, 2H); 3.15(dd, 1H); 3.00-2.90(m, 1H); 2.74-2.63(m, 1H); 2.72(dd, 1H); 2.60-2.48(m, 1H); 2.41-2.21(m, 2H); 1.94-1.79(m, 2H); 1.38-1.23(m, 2H). MS: [ES MS] m/z 436(MH+) | 0.102 | Prepn. 2 | (2E)-3-(3- fluoro- phenyl)- 2-propenal Preparation 5 |
| 3 | 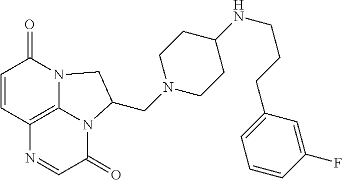 | 2-[(4-{[3-(3- fluorophenyl)propyl] amino}-1- piperidinyl)methyl]- l,2-dihydro-3H,8H- 2a,5,8a- triazaacenaphthylene- 3,8-dione | 1H-NMR(δ, ppm, CDCl₃): 7.83(s, 1H); 7.77(d, 1H); 7.27-7.20(m, 1H); 6.99-6.84(m, 3H); 6.39(d, 1H); 5.07-4.99(m, 1H); 4.55(dd, 1H); 4.38(dd, 1H); 3.13 (dd, 1H); 2.98-2.88(m, 1H); 2.74-2.61(m, 6H); 2.51-2.39(m, 1H); 2.37-2.20(m, 2H); l .89-1.74(m, 2H); 1.37-1.22(m, 2H). MS: [ES MS] m/z 438(MH+) | 0.82 | Prepn. 2 | 3-(3- fluoro- phenyl) propanal Preparation 6 |
| 4 | 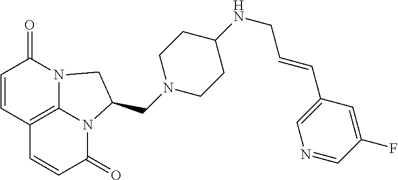 | (1R)-1-[(4-{[(2E)-3-(5- fluoro-3-pyridinyl)-2- propen-1-yl]amino}-1- piperidinyl)methyl]- 1,2-dihydro-4H,9H- imidazo[l,2,3-ij]-1,8- naphthyridine- 4,9-dione | 1H-NMR(δ, ppm,CDCl₃): 8.39(bs, 1H); 8.31(d, 1H); 7.49(dd, 2H); 7.39(dt, 1H); 6.56-6.50(m, 1H); 6.43-6.34(m, 1H); 6.27(dd,2H); 5.05-4.97(m, 1H); 4.57(dd, 1H); 4.35(dd, 1H); 3.46(dd, 2H); 3.10(dd, 1H); 3.01-2.93(m, 1H); 2.68(dd, 2H); 2.58-2.46(m, 1H); 2.38-2.20(m, 2H); 1.91-1.81(m, 2H); 1.36-1.28(m, 2H). MS: [ES MS] m/z 436(MH+) | 0.544 | Prepn. 1 | (2E)-3- (5-fluoro-3- pyridinyl)- 2-propenal Preparation 7 |
| 5 | 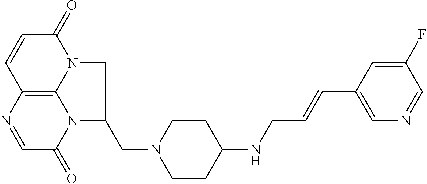 | 2-[(4-{[(2E)-3-(5- fluoro-3-pyridinyl)-2- propen-1-yl]amino}-1- piperidinyl)methyl]- 1,2-dihydro-3H,8H- 2a,5,8a- triazaacenaphthylene- 3,8-dione | 1H-NMR(δ, ppm,CDCl₃): 8.40(bs, 1H); 8.33(d, 1H); 7.84(s, 1H); 7.40(dt, 1H); 6.54(d,1H); 6.44-6.34(m, 2H); 5.09-4.99(m ,1H); 4.56(dd, 1H); 4.39(dd, 1H); 3.46(dd, 2H); 3.15(dd, 1H); 2.99-2.92(m, 1H); 2.76-2.66(m, 2H); 2.59-2.49(m, 1H); 2.41-2.24(m, 2H); 1.93-1.82(m, 2H); 1.37-1.25(m, 2H). MS: [ES MS] m/z 437 (MH+) | 0.273 | Prepn. 2 | (2E)-3- (5-fluoro-3- pyridinyl)- 2-propenal Preparation 7 |
| 6 | 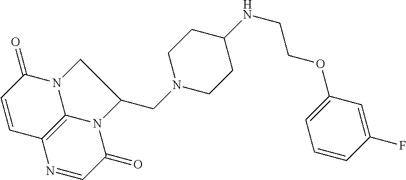 | 2-{[4-({2-[(3- fluorophenyl)oxy]ethyl} amino)-1- piperidinyl]methyl}- l,2-dihydro-3H,8H- 2a,5,8a- triazaacenaphthylene- 3,8-dione | 1H-NMR(δ, ppm,CDCl₃): 7.83(s, 1H); 7.76(d, 1H); 7.22-7.17(m, 1H); 6.70-6.59(m, 3H); 6.38(d, 1H); 5.08-4.99(m, 1H); 4.58-4.52(m, 1H); 4.42-4.34(m, 1H); 4.04(t, 2H); 3.17-3.11(m, 1H); 2.99(t, 2H); 2.95-2.92(m, 1H); 2.75-2.67(m, 2H); 2.56-2.48(m, 1H); 2.39-2.23(m, 2H); 1.90-1.80(m, 2H); 1.37-1.25(m, 2H). MS: [ES MS] m/z 440 (MH+) | 0.824 | Prepn. 2 | [(3-fluoro- phenyl)oxy] acetaldehyde Preparation 8 |
| 7 | 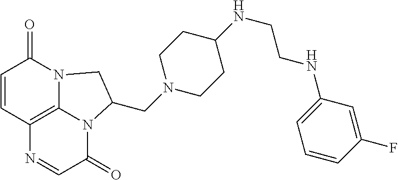 | 2-{[4-({2-[(3- fluorophenyl)amino] ethyl}amino)-1- piperidinyl]methyl}- l,2-dihydro-3H,8H- 2a,5,8a- triazaacenaphthylene- 3,8-dione | 1H-NMR(δ, ppm, CDCl₃): 7.83-7.74(m, 2H); 7.13-7.04(m, 1H); 6.44-6.29(m, 4H), 5.08-4.97(m, 1H); 4.52(dd, 1H); 4.39(dd, 1H); 3.29(bs, 2H); 3.16(dd, 1H); 3.01-2.93(m, 3H); 2.75-2.55(m, 3H); 2.38-2.19(m, 2H); 1.98-1.83(m, 2H); 1.54-1.38(m, 2H). MS: [ES MS] m/z 439(MH+) | 0.071 | Prepn. 2 | 2-[(3- fluorophenyl) amino]ethyl methane- sulfonate Preparation 9 |
| 8 | 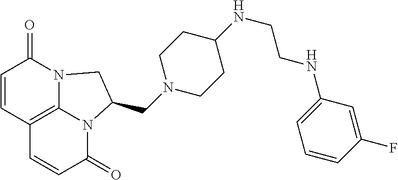 | (1R)-1-{[4-({2-[(3- fluorophenyl)amino] ethyl}amino)-1- piperidinyl]methyl}- l,2-dihydro-4H,9H- imidazo[l,2,3-ij]-1,8- naphthyridine- 4,9-dione | 1H-NMR(δ, ppm, CDCl₃): 7.49(dd, 2H); 7.08(q, 1H); 6.44-6.21(m, 5H), 5.04-4.94(m, 1H); 4.54(dd, 1H); 4.35(dd, 1H); 3.30(bs, 2H); 3.10(dd, 1H); 3.02-2.91(m, 3H); 2.73-2.58(m, 3H); 2.36-2.16(m, 2H); 1.99-1.83(m, 2H); 1.49-1.37(m, 2H). MS: [ES MS] m/z 438(MH+) | See summary | Prepn. 1 | 2-[(3- fluorophenyl) amino] ethyl methane- sulfonate Preparation 9 |
| 9 | 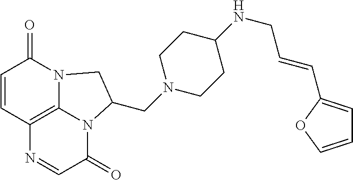 | 2-[(4-{[(2E)-3-(2- furanyl)-2-propen-1- yl]amino}-1- piperidinyl)methyl]- 1,2-dihydro-3H,8H- 2a,5,8a- triazaacenaphthylene- 3,8-dione | 1H-NMR(d, ppm,CDC1₃): 7.82(s, 1H); 7.76(d, 1H); 7.33(s, 1H); 6.45-6.30(m, 3H), 6.28-6.13(m, 2H); 5.13-4.93(m, 1H); 4.55(dd, 1H); 4.37(dd, 1H); 3.41 (d, 2H); 3.14(dd, 1H); 2.93(bd, 1H); 2.76-2.62(m, 2H); 2.62-1.7(m, 1H); 2.41-2.19(m, 2H); 1.94-1.79(m, 2H); 1.43-1.29(m, 2H). | See summary | As Example 1 | (2E)-3-(2- furanyl)-2- propenal (Commercial) |
| 10 | 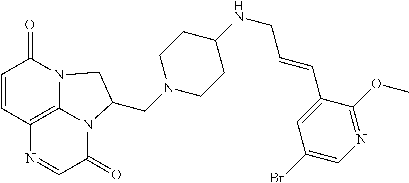 | 2-{[4-({(2E)-3-[5- bromo-2-(methyloxy)- 3-pyridinyl]-2-propen- 1-yl}amino)-1- piperidinyl]methyl}- 1,2-dihydro-3H,8H- 2a,5,8a- triazaacenaphthylene- 3,8-dione | 1H-NMR(d, ppm, CDCl₃): 8.07(d, 1H); 7.85-7.71 (m, 3H); 6.64(d, 1H); 6.48-6.33(m, 2H); 5.10-4.97(m, 1H); 4.60-4.34(m, 2H); 3.95(t, 3H); 3.49(d, 2H); 3.15(dd, 1H); 2.97(bd, 1H); 2.79-2.65(m, 3H); 2.44-2.17(m, 2H); 2.00-1.82(m, 2H); 1.34-1.23(m, 2H) | See summary | As Example 1 | (2E)-3-[5- bromo-2- (methyloxy)-3- pyridinyl]- 2-propenal (Preparation 10) |
| 11 | 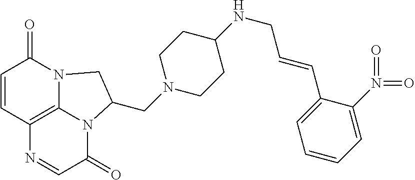 | 2-[(4-{[(2E)-3-(2- nitrophenyl)-2-propen- 1-yl]amino}-1- piperidinyl)methyl]- l,2-dihydro-3H,8H- 2a,5,8a- triazaacenaphthylene- 3,8-dione | 1H-NMR(d, ppm, CDC1₃): 7.93(d, 1H); 7.84(s, 1H);7.78(d, 1H); 7.63-7.53(m, 2H); 7.43-7.36(m, 1H); 7.0(d, 1H); 6.40(d, 1H); 6.32-6.22(m, 1H); 5.09-5.0(m, 1H); 4.57(dd, 1H); 4.39(dd, 1H); 3.50(dd, 2H); 3.15(dd, 1H); 3.01-2.93(m, 1H); 2.76-2.66(m, 2H); 2.64-2.53(m, 1H); 2.43-2.24(m, 2H); 1.97-1.82(m, 2H); 1.39-1.29(m, 2H). | See summary | As Example 1 | (2E)-3-(2- nitrophenyl)- 2-propenal (Commercial) |
| 12 | 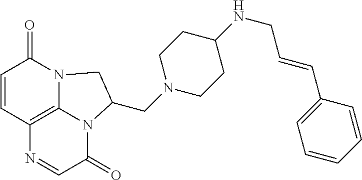 | 2-[(4-{[(2E)-3-phenyl- 2-propen-1-yl]amino}- 1-piperidinyl)methyl]- 1,2-dihydro-3H,8H- 2a,5,8a- triazaacenaphthylene- 3,8-dione | 1H-NMR(d, ppm, CDCl₃): 7.77(d, 1H); 7.17-7.42(m, 5H); 7.76(d, 1H);; 6.54(d, 1H), 6.39(d, 1H); 6.35-6.23(m, 1H); 5.08-4.96(m, 1H); 4.55(dd, 1H); 4.37(dd, 1H); 3.34(d, 2H); 3.13(dd, 1H); 2.94(bd, 1H); 2.75-2.34(m, 3H); 2.40-1.84(m, 2H); 1.98-1.81 (m, 2H); 1.46-1.30(m, 2H). | See summary | As Example 1 | (2E)-3- phenyl-2- propenal (Commercial) |
| 13 | 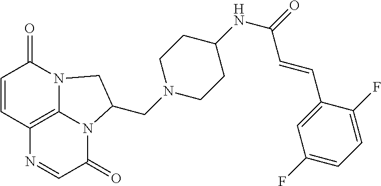 | (2E)-3-(2,5- difluorophenyl)-N-{1- [(3,8-dioxo-1,2- dihydro-3H,8H- 2a,5,8a- triazaacenaphthylen-2- yl)methyl]-4- piperidinyl}-2- propenamide | 1H-NMR(d, ppm, CD3OD): 7.91(d, 1H); 7.82(s, 1H); 7.46-7.39(m, 2H); 7.32(d, 1H); 7.27-7.17(m, 2H); 6.40(d, 1H); 5.28-5.19(m, 1H); 4.50(dd, 1H); 4.22(s, 2H); 3.27-3.25(m, 1H); 3.13-2.99(m, 2H); 2.58-2.46(m, 2H); 2.21-2.12(m, 2H); 1.76-1.59(m, 2H). | See summary | As Example 1 | trans-2,5- difluoro- cinnamic acid (Commercial) |
| 14 | 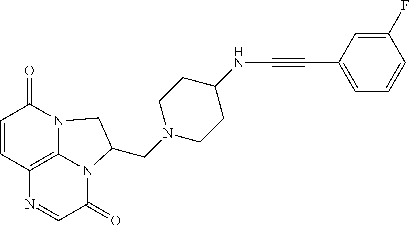 | 2-[(4-{[(3- fluorophenyl)ethynyl] amino}-1- piperidinyl)methyl]- 1,2-dihydro-3H,8H- 2a,5,8a- triazaacenaphthylene- 3,8-dione | 1H-NMR(d, ppm, CD3OD): 7.91(d, 1H); 7.82(s, 1H); 7.46-7.39(m, 2H); 7.32(d, 1H); 7.27-7.17(m, 2H); 6.40(d, 1H); 5.28-5.19(m, 1H); 4.50(dd, 1H); 4.22(s, 2H); 3.27-3.25(m, 1H); 3.13-2.99(m, 2H); 2.58-2.46(m, 2H); 2.21-2.12(m, 2H); 1.76-1.59(m, 2H). | See summary | As Example 1 | 3-(3- fluorophenyl)- 2-propynal (See preparation 11) |
| 15 | 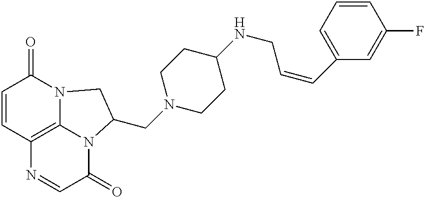 | 2-[(4-{[(2Z)-3-(3- fluorophenyl)-2- propen-1-yl]amino}-1 piperidinyl)methyl]- 1,2-dihydro-3H,8H- 2a,5,8a- triazaacenaphthylene- 3,8-dione | 1H-NMR(d, ppm, CDCl₃): 7.82(s, 1H); 7.76(d, 1H); 7.34-7.26(m, 1H); 7.00-6.93(m, 2H); 6.54-6.50(d, 1H), 6.39-6.36(d, 1H); 5.85-5.77(m, 1H); 5.06-4.97(m, 1H); 4.57-4.50(m, 1H); 4.41-4.33(m, 1H); 3.56-3.54(d, 1H); 3.17-3.10(m, 1H); 2.95-2.90(m, 1H); 2.74-2.64(m, 2H); 2.59-2.49(m, 1H); 2.35-2.18(m, 2H); 1.85-1.62(m, 4H); 1.41-1.31(m, 2H). | See summary | As Example 1 | (2Z)-3-(3- fluorophenyl)- 2-propenal (See preparation 12) |
| 16 | 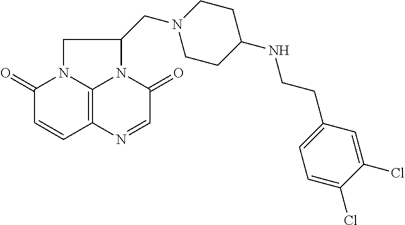 | 2-[(4-{[2-(3,4- dichlorophenyl)ethyl] amino}-1- piperidinyl)methyl]- 1,2-dihydro-3H,8H- 2a,5,8a- triazaacenaphthylene- 3,8-dione | 1H-NMR(d, ppm, CD3OD): 7.82(s, 1H); 7.76(d, 1H); 7.35(d, 1H); 7.29(d, 1H); 7.04(dd, 1H); 6.37(d, 1H); 5.08-4.97(m, 1H); 4.53(dd, 1H); 4.37(dd, 1H); 3.13(dd, 1H); 2.99-2.83(m, 3H); 2.82-2.75(m, 4H); 2.57-2.44(m, 1H); 2.19-2.38(m, 2H), l.90-l.71(m, 2H); 1.41-1.02(m, 2H). | See summary | | 2-(3,4- dichloro- phenyl) ethyl methane- sulfonate (See preparation 13) |
| 17 | 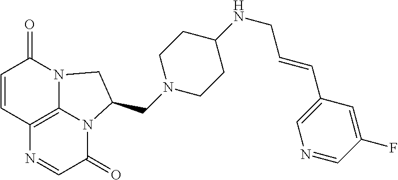 | (2R)-2-[(4-{[(2E)-3-(5- fluoro-3-pyridinyl)-2- propen-1-yl]amino}-1- piperidinyl)methyl]- 1,2-dihydro-3H,8H- 2a,5,8a- triazaacenaphthylene- 3,8-dione | 1H-NMR(d, ppm, CDCl₃): 8.39(s, 1H); 8.32(d, 1H); 7.83(s, 1H); 7.77(d, 1H); 7.41-7.36(m, 1H); 6.53(d, 1H), 6.43-6.33(m, 2H); 5.08-4.99(m, 1H); 4.56(dd, 1H); 4.42-4.35(m, 1H); 3.46(d, 2H); 3.15(dd, 1H); 2.98-2.92(m, 1H); 2.75-2.66(m, 2H); 2.58-2.48(m, 1H); 2.40-2.23(m, 2H); 1.92-1.80(m, 2H); 1.39-1.25(m, 2H). | See summary | As Example 1 | (2E)-3-(5- fluoro-3- pyridinyl)-2- propenal (See preparation 7) |

1. A compound of Formula (I) or a pharmaceutically acceptable salt or N-oxide thereof: 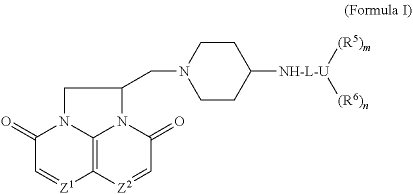
(relative stereochemistry shown)
wherein:
one of Z¹ and Z² is CH or N and the other is CH;
L is selected from: —CH₂—CH═CH—, —CH═CH—CH₂—, —(CH₂)ₚ— where p is 2, 3 or 4, —CH₂—CH₂—O—, —O—CH₂—CH₂—, —CH₂—CH₂—NH—, —HN—CH₂—CH₂—, —C(O)—CH═CH—, —CH═CH—C(O)—, —CH═CH, or —C≡C—;
U represents a cyclic group selected from: phenyl, pyridyl, pyridazinyl, pyrimidinyl, pyrazinyl, thiazolyl, furanyl, imidazolyl and thiophenyl;
m is 0 or 1,
n is independently 0 or 1; and
substituent(s) R⁵ and R⁶ are independently selected from: halo, CF₃, OCF₃, C₁₋₃ alkyl, C₁₋₃ alkoxy, nitro and cyano.

2. A compound of claim 1 wherein the compound of Formula (I) is a compound of Formula (IA) or a pharmaceutically acceptable salt or N-oxide thereof: 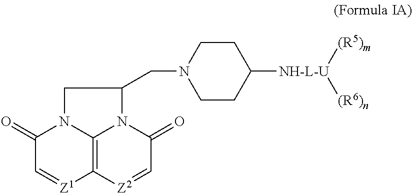
(relative stereochemistry shown)
wherein:
one of Z¹ and Z² is CH or N and the other is CH;
L is selected from: —CH₂—CH═CH—, —CH═CH—CH₂—, —(CH₂)ₚ— where p is 2, 3 or 4, —CH₂—CH₂—O—, —O—CH₂—CH₂—, —CH₂—CH₂—NH—, —HN—CH₂—CH₂—, —C(O)—CH═CH— or —CH═CH—C(O)—;
U represents a cyclic group selected from: phenyl, pyridyl, pyridazinyl, pyrimidinyl, pyrazinyl, thiazolyl, furanyl, imidazolyl and thiophenyl;
m is 0 or 1,
n is independently 0 or 1; and
substituent(s) R⁵ and R⁶ are independently selected from: halo, CF₃, OCF₃, C₁₋₃ alkyl, C₁₋₃ alkoxy, nitro and cyano.

3. A compound of claim 1 or a pharmaceutically acceptable salt or N-oxide thereof wherein cyclic group U is phenyl, 3-pyridyl or 2-furanyl.

4. A compound of claim 1 or a pharmaceutically acceptable salt or N-oxide thereof wherein in lnker L, p is 3.

5. A compound of claim 1 or a pharmaceutically acceptable salt or N-oxide thereof wherein C₁₋₃ alkoxy is methoxy.

6. A compound of claim 1 or a pharmaceutically acceptable salt or N-oxide thereof wherein R⁵ is fluorine, m is 1 and n is 0.

7. A compound of claim 1 or a pharmaceutically acceptable salt or N-oxide thereof wherein m is 1, n is 1 R⁵ is fluorine and R⁶ is also fluorine.

8. A compound of claim 1 or a pharmaceutically acceptable salt or N-oxide thereof wherein m is 1, n is 0 and R⁵ is NO₂.

9. A compound of claim 1 or a pharmaceutically acceptable salt or N-oxide thereof wherein m is 1, n is 1 R⁵ is bromine and R⁶ is methoxy.

10. A compound of claim 1 or a pharmaceutically acceptable salt or N-oxide thereof wherein the absolute stereochemistry is indicated by the Formula (IB):

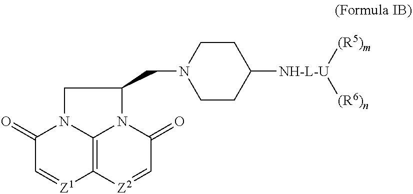

11. A compound of Formula (I) as claimed in claim 1 selected from the group consisting of:
(1R)-1-[(4-{[(2E)-3-(2,5-difluorophenyl)-2-propen-1-yl]amino}-1-piperidinyl)methyl]-1,2-dihydro-4H,9H-imidazo[1,2,3-ij]-1,8-naphthyridine-4,9-dione;
2-[(4-{[(2E)-3-(3-fluorophenyl)-2-propen-1-yl]amino}-1-piperidinyl)methyl]-1,2-dihydro-3H,8H-2a,5,8a-triazaacenaphthylene-3,8-dione;
2-[(4-{[3-(3-fluorophenyl)propyl]amino}-1-piperidinyl)methyl]-1,2-dihydro-3H,8H-2a,5,8a-triazaacenaphthylene-3,8-dione;
(1R)-1-[(4-{[(2E)-3-(5-fluoro-3-pyridinyl)-2-propen-1-yl]amino}-1-piperidinyl)methyl]-1,2-dihydro-4H,9H-imidazo[1,2,3-ij]-1,8-naphthyridine-4,9-dione;
2-[(4-{[(2E)-3-(5-fluoro-3-pyridinyl)-2-propen-1-yl]amino}-1-piperidinyl)methyl]-1,2-dihydro-3H,8H-2a,5,8a-triazaacenaphthylene-3,8-dione;
2-{[4-({2-[(3 -fluorophenyl)oxy]ethyl}amino)-1-piperidinyl]methyl}-1,2-dihydro-3H,8H-2a,5,8a-triazaacenaphthylene-3,8-dione;
2-{[4-({2-[(3-fluorophenyl)amino]ethyl}amino)-1-piperidinyl]methyl}-1,2-dihydro-3H,8H-2a,5,8a-triazaacenaphthylene-3,8-dione;
(1R)-1-{[4-({2-[(3-fluorophenyl)amino]ethyl}amino)-1-piperidinyl]methyl}-1,2-dihydro-4H,9H-imidazo[1,2,3-ij]-1 ,8-naphthyridine-4,9-dione;
2-[(4-{[(2E)-3-(2-furanyl)-2-propen-1-yl]amino}-1-piperidinyl)methyl]-1,2-dihydro-3H,8H-2a,5,8a-triazaacenaphthylene-3,8-dione;
2-{[4-({(2E)-3-[5-bromo-2-(methyloxy)-3-pyridinyl]-2-propen-1-yl}amino)-1-piperidinyl]methyl}-1,2-dihydro-3H,8H-2a,5,8a-triazaacenaphthylene-3,8 -dione;
2-[(4-{[(2E)-3-(2-nitrophenyl)-2-propen-1-yl]amino}-1-piperidinyl)methyl]-1,2-dihydro-3H,8H-2a,5,8a-triazaacenaphthylene-3,8-dione;
2-[(4-{[(2E)-3-phenyl-2-propen-1-yl]amino}-1-piperidinyl)methyl]-1,2-dihydro-3H,8H-2a,5,8a-triazaacenaphthylene-3,8-dione;
(2E)-3-(2,5-difluorophenyl)-N-{1-[(3,8-dioxo-1,2-dihydro-3H,8H-2a,5,8a-triazaacenaphthylen-2-yl)methyl]-4-piperidinyl}-2-propenamide;
2-[(4-{[(3-fluorophenyl)ethynyl]amino}-1-piperidinyl)methyl]-1,2-dihydro-3H,8H-2a,5,8a-triazaacenaphthylene-3,8-dione;
2-[(4-{[(2Z)-3-(3-fluorophenyl)-2-propen-1-yl]amino}-1-piperidinyl)methyl]-1,2-dihydro-3H,8H-2a,5,8a-triazaacenaphthylene-3,8-dione;
2-[(4-{[2-(3,4-dichlorophenyl)ethyl]amino}-1-piperidinyl)methyl]-1,2-dihydro-3H,8H-2a,5,8a-triazaacenaphthylene-3,8-dione; and;
(2R)-2-[(4-{[(2E)-3-(5-fluoro-3-pyridinyl)-2-propen-1-yl]amino}-1-piperidinyl)methyl]-1,2-dihydro-3H,8H-2a,5,8a-triazaacenaphthylene-3,8-dione.

12. A pharmaceutical composition comprising a compound of Formula (I), or a pharmaceutically acceptable salt or N-oxide thereof, as claimed in claim 1, and one or more pharmaceutically acceptable carriers, excipients or diluents.

13. A method of treatment of tuberculosis in mammals, particularly in man, which method comprises the administration to a mammal in need of such treatment an effective amount of a compound of Formula (I), or a pharmaceutically acceptable salt or N-oxide thereof, as claimed in claim 1.

14. A method of treatment of bacterial infections in mammals, particularly in man, which method comprises the administration to a mammal in need of such treatment an effective amount of a compound of Formula (I), or a pharmaceutically acceptable salt or N-oxide thereof, as claimed in claim 1.

15. A compound of Formula (I), or a pharmaceutically acceptable salt or N-oxide thereof, as claimed in claim 1, for use in therapy.

16. A compound of Formula (I), or a pharmaceutically acceptable salt or N-oxide thereof, as claimed in claim 1, for use in the treatment of tuberculosis in mammals.

17. A compound of Formula (I), or a pharmaceutically acceptable salt or N-oxide thereof, as claimed in claim 1, for use in the treatment of bacterial infections in mammals.

18. The use of a compound of Formula (I), or a pharmaceutically acceptable salt or N-oxide thereof, as claimed in claim 1, in the manufacture of a medicament for use in the treatment of tuberculosis in mammals.

19. The use of a compound of Formula (I), or a pharmaceutically acceptable salt or N-oxide thereof, as claimed in claim 1, in the manufacture of a medicament for use in the treatment of bacterial infections in mammals.

20. A pharmaceutical composition comprising a compound of Formula (I), or a pharmaceutically acceptable salt or N-oxide thereof, as claimed in claim 1, and one or more pharmaceutically acceptable carriers, excipients or diluents, for use in the treatment of tuberculosis in mammals.

21. A pharmaceutical composition comprising a compound of Formula (I), or a pharmaceutically acceptable salt or N-oxide thereof, as claimed in claim 1, and one or more pharmaceutically acceptable carriers, excipients or diluents, for use in the treatment of bacterial infections in mammals.

22. A process for making a compound of Formula (I), or a pharmaceutically acceptable salt or N-oxide thereof, as claimed in claim 1, comprising the reaction between an amine compound of Formula (IIA) and a compound of Formula (IIB): 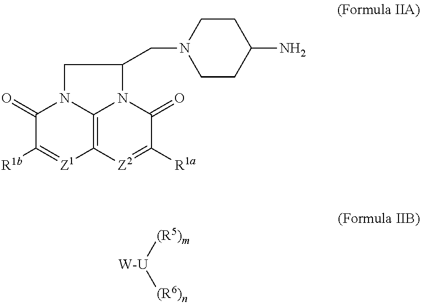
wherein Z¹, Z², L, U, m, n, R⁵ and R⁶ are as defined in Formula (I), and W is a moiety which can react with the —NH₂ group to form the moiety —NH-L-.
